SEQUENCE LISTING (99 sequences)

SEQ ID NO: 1 - protein, 94 aa, Homo sapiens
SCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 2 - protein, 102 aa, Artificial
  PSP94 family member
EAEAYVEFSCYFIPNEGVPGDSTRKCMDLKGNKHPINSEWQTDNCETCTCYETEISCCTLVSTPVGYDKDNCQRIFKKEDCKYIVVEKKDPKKTCSVSEWII

SEQ ID NO: 3 - protein, 10 aa, Artificial
  PSP94 family member
YTCSVSEPGI

SEQ ID NO: 4 - protein, 15 aa, Artificial
  PSP94 family member
NEGVPGDSTRKCMDL

SEQ ID NO: 5 - protein, 15 aa, Artificial
  PSP94 family member
EWQTDNCETCTCYET

SEQ ID NO: 6 - protein, 19 aa, Artificial
  PSP94 family member
IVVEKKDPKKTCSVSEWII

SEQ ID NO: 7 - protein, 15 aa, Artificial
  PCK3145 derivative
EWQTDNCETCTCYET

SEQ ID NO: 8 - DNA, 285 bp, Artificial
  cDNA
tcatgctatttcatacctaatgagggagttccaggagattcaaccaggaaatgcatggatctcaaaggaaacaaacacccaataaactcggagtggcagactgacaactgtgagacatgcacttgctacgaaacagaaatttcatgttgcacccttgtttctacacctgtgggttatgacaaagacaactgccaaagaatcttcaagaaggaggactgcaagtatatcgtggtggagaagaaggacccaaaaaagacctgttctgtcagtgaatggataatctaa

SEQ ID NO: 9 - protein, 16 aa, Artificial
  PCK3145 derivative
EWQTDNCETCTCYETE

SEQ ID NO: 10 - protein, 17 aa, Artificial
  PCK3145 derivative
EWQTDNCETCTCYETEI

SEQ ID NO: 11 - protein, 18 aa, Artificial
  PCK3145 derivative
EWQTDNCETCTCYETEIS

SEQ ID NO: 12 - protein, 19 aa, Artificial
  PCK3145 derivative
EWQTDNCETCTCYETEISC

SEQ ID NO: 13 - protein, 20 aa, Artificial
  PCK3145 derivative
EWQTDNCETCTCYETEISCC

SEQ ID NO: 14 - protein, 21 aa, Artificial
  PCK3145 derivative
EWQTDNCETCTCYETEISCCT

SEQ ID NO: 15 - protein, 22 aa, Artificial
  PCK3145 derivative
EWQTDNCETCTCYETEISCCTL

SEQ ID NO: 16 - protein, 23 aa, Artificial
  PCK3145 derivative
EWQTDNCETCTCYETEISCCTLV

SEQ ID NO: 17 - protein, 24 aa, Artificial
  PCK3145 derivative
EWQTDNCETCTCYETEISCCTLVS

SEQ ID NO: 18 - protein, 25 aa, Artificial
  PCK3145 derivative
EWQTDNCETCTCYETEISCCTLVST

SEQ ID NO: 19 - protein, 26 aa, Artificial
  PCK3145 derivative
EWQTDNCETCTCYETEISCCTLVSTP

SEQ ID NO: 20 - protein, 27 aa, Artificial
  PCK3145 derivative
EWQTDNCETCTCYETEISCCTLVSTPV

SEQ ID NO: 21 - protein, 28 aa, Artificial
  PCK3145 derivative
EWQTDNCETCTCYETEISCCTLVSTPVG

SEQ ID NO: 22 - protein, 29 aa, Artificial
  PCK3145 derivative
EWQTDNCETCTCYETEISCCTLVSTPVGY

SEQ ID NO: 23 - protein, 30 aa, Artificial
  PCK3145 derivative
EWQTDNCETCTCYETEISCCTLVSTPVGYD

SEQ ID NO: 24 - protein, 31 aa, Artificial
  PCK3145 derivative
EWQTDNCETCTCYETEISCCTLVSTPVGYDK

SEQ ID NO: 25 - protein, 32 aa, Artificial
  PCK3145 derivative
EWQTDNCETCTCYETEISCCTLVSTPVGYDKD

SEQ ID NO: 26 - protein, 33 aa, Artificial
  PCK3145 derivative
EWQTDNCETCTCYETEISCCTLVSTPVGYDKDN

SEQ ID NO: 27 - protein, 34 aa, Artificial
  PCK3145 derivative
EWQTDNCETCTCYETEISCCTLVSTPVGYDKDNC

SEQ ID NO: 28 - protein, 35 aa, Artificial
  PCK3145 derivative
EWQTDNCETCTCYETEISCCTLVSTPVGYDKDNCQ

SEQ ID NO: 29 - protein, 36 aa, Artificial
  PCK3145 derivative
EWQTDNCETCTCYETEISCCTLVSTPVGYDKDNCQR

SEQ ID NO: 30 - protein, 37 aa, Artificial
  PCK3145 derivative
EWQTDNCETCTCYETEISCCTLVSTPVGYDKDNCQRI

SEQ ID NO: 31 - protein, 38 aa, Artificial
  PCK3145 derivative
EWQTDNCETCTCYETEISCCTLVSTPVGYDKDNCQRIF

SEQ ID NO: 32 - protein, 39 aa, Artificial
  PCK3145 derivative
EWQTDNCETCTCYETEISCCTLVSTPVGYDKDNCQRIFK

SEQ ID NO: 33 - protein, 40 aa, Artificial
  PCK3145 derivative
EWQTDNCETCTCYETEISCCTLVSTPVGYDKDNCQRIFKK

SEQ ID NO: 34 - protein, 41 aa, Artificial
  PCK3145 derivative
EWQTDNCETCTCYETEISCCTLVSTPVGYDKDNCQRIFKKE

SEQ ID NO: 35 - protein, 42 aa, Artificial
  PCK3145 derivative
EWQTDNCETCTCYETEISCCTLVSTPVGYDKDNCQRIFKKED

SEQ ID NO: 36 - protein, 43 aa, Artificial
  PCK3145 derivative
EWQTDNCETCTCYETEISCCTLVSTPVGYDKDNCQRIFKKEDC

SEQ ID NO: 37 - protein, 44 aa, Artificial
  PCK3145 derivative
EWQTDNCETCTCYETEISCCTLVSTPVGYDKDNCQRIFKKEDCK

SEQ ID NO: 38 - protein, 45 aa, Artificial
  PCK3145 derivative
EWQTDNCETCTCYETEISCCTLVSTPVGYDKDNCQRIFKKEDCKY

SEQ ID NO: 39 - protein, 46 aa, Artificial
  PCK3145 derivative
EWQTDNCETCTCYETEISCCTLVSTPVGYDKDNCQRIFKKEDCKYI

SEQ ID NO: 40 - protein, 47 aa, Artificial
  PCK3145 derivative
EWQTDNCETCTCYETEISCCTLVSTPVGYDKDNCQRIFKKEDCKYIV

SEQ ID NO: 41 - protein, 48 aa, Artificial
  PCK3145 derivative
EWQTDNCETCTCYETEISCCTLVSTPVGYDKDNCQRIFKKEDCKYIVV

SEQ ID NO: 42 - protein, 49 aa, Artificial
  PCK3145 derivative
EWQTDNCETCTCYETEISCCTLVSTPVGYDKDNCQRIFKKEDCKYIVVE

SEQ ID NO: 43 - protein, 50 aa, Artificial
  PCK3145 derivative
EWQTDNCETCTCYETEISCCTLVSTPVGYDKDNCQRIFKKEDCKYIVVEK

SEQ ID NO: 44 - protein, 51 aa, Artificial
  PCK3145 derivative
EWQTDNCETCTCYETEISCCTLVSTPVGYDKDNCQRIFKKEDCKYIVVEKK

SEQ ID NO: 45 - protein, 52 aa, Artificial
  PCK3145 derivative
EWQTDNCETCTCYETEISCCTLVSTPVGYDKDNCQRIFKKEDCKYIVVEKKD

SEQ ID NO: 46 - protein, 53 aa, Artificial
  PCK3145 derivative
EWQTDNCETCTCYETEISCCTLVSTPVGYDKDNCQRIFKKEDCKYIVVEKKDP

SEQ ID NO: 47 - protein, 54 aa, Artificial
  PCK3145 derivative
EWQTDNCETCTCYETEISCCTLVSTPVGYDKDNCQRIFKKEDCKYIVVEKKDPK

SEQ ID NO: 48 - protein, 55 aa, Artificial
  PCK3145 derivative
EWQTDNCETCTCYETEISCCTLVSTPVGYDKDNCQRIFKKEDCKYIVVEKKDPKK

SEQ ID NO: 49 - protein, 56 aa, Artificial
  PCK3145 derivative
EWQTDNCETCTCYETEISCCTLVSTPVGYDKDNCQRIFKKEDCKYIVVEKKDPKKT

SEQ ID NO: 50 - protein, 57 aa, Artificial
  PCK3145 derivative
EWQTDNCETCTCYETEISCCTLVSTPVGYDKDNCQRIFKKEDCKYIVVEKKDPKKTC

SEQ ID NO: 51 - protein, 58 aa, Artificial
  PCK3145 derivative
EWQTDNCETCTCYETEISCCTLVSTPVGYDKDNCQRIFKKEDCKYIVVEKKDPKKTCS

SEQ ID NO: 52 - protein, 59 aa, Artificial
  PCK3145 derivative
EWQTDNCETCTCYETEISCCTLVSTPVGYDKDNCQRIFKKEDCKYIVVEKKDPKKTCSV

SEQ ID NO: 53 - protein, 60 aa, Artificial
  PCK3145 derivative
EWQTDNCETCTCYETEISCCTLVSTPVGYDKDNCQRIFKKEDCKYIVVEKKDPKKTCSVS

SEQ ID NO: 54 - protein, 61 aa, Artificial
  PCK3145 derivative
EWQTDNCETCTCYETEISCCTLVSTPVGYDKDNCQRIFKKEDCKYIVVEKKDPKKTCSVSE

SEQ ID NO: 55 - protein, 62 aa, Artificial
  PCK3145 derivative
EWQTDNCETCTCYETEISCCTLVSTPVGYDKDNCQRIFKKEDCKYIVVEKKDPKKTCSVSEW

SEQ ID NO: 56 - protein, 63 aa, Artificial
  PCK3145 derivative
EWQTDNCETCTCYETEISCCTLVSTPVGYDKDNCQRIFKKEDCKYIVVEKKDPKKTCSVSEWI

SEQ ID NO: 57 - protein, 64 aa, Artificial
  PCK3145 derivative
EWQTDNCETCTCYETEISCCTLVSTPVGYDKDNCQRIFKKEDCKYIVVEKKDPKKTCSVSEWII

SEQ ID NO: 58 - protein, 16 aa, Artificial
  PCK3145 derivative
SEWQTDNCETCTCYET

SEQ ID NO: 59 - protein, 17 aa, Artificial
  PCK3145 derivative
NSEWQTDNCETCTCYET

SEQ ID NO: 60 - protein, 18 aa, Artificial
  PCK3145 derivative
INSEWQTDNCETCTCYET

SEQ ID NO: 61 - protein, 19 aa, Artificial
  PCK3145 derivative
PINSEWQTDNCETCTCYET

SEQ ID NO: 62 - protein, 20 aa, Artificial
  PCK3145 derivative
HPINSEWQTDNCETCTCYET

SEQ ID NO: 63 - protein, 21 aa, Artificial
  PCK3145 derivative
KHPINSEWQTDNCETCTCYET

SEQ ID NO: 64 - protein, 22 aa, Artificial
  PCK3145 derivative
NKHPINSEWQTDNCETCTCYET

SEQ ID NO: 65 - protein, 23 aa, Artificial
  PCK3145 derivative
GNKHPINSEWQTDNCETCTCYET

SEQ ID NO: 66 - protein, 24 aa, Artificial
  PCK3145 derivative
KGNKHPINSEWQTDNCETCTCYET

SEQ ID NO: 67 - protein, 25 aa, Artificial
  PCK3145 derivative
LKGNKHPINSEWQTDNCETCTCYET

SEQ ID NO: 68 - protein, 26 aa, Artificial
  PCK3145 derivative
DLKGNKHPINSEWQTDNCETCTCYET

SEQ ID NO: 69 - protein, 27 aa, Artificial
  PCK3145 derivative
MDLKGNKHPINSEWQTDNCETCTCYET

SEQ ID NO: 70 - protein, 28 aa, Artificial
  PCK3145 derivative
CMDLKGNKHPINSEWQTDNCETCTCYET

SEQ ID NO: 71 - protein, 29 aa, Artificial
  PCK3145 derivative
KCMDLKGNKHPINSEWQTDNCETCTCYET

SEQ ID NO: 72 - protein, 30 aa, Artificial
  PCK3145 derivative
RKCMDLKGNKHPINSEWQTDNCETCTCYET

SEQ ID NO: 73 - protein, 31 aa, Artificial
  PCK3145 derivative
TRKCMDLKGNKHPINSEWQTDNCETCTCYET

SEQ ID NO: 74 - protein, 32 aa, Artificial
  PCK3145 derivative
STRKCMDLKGNKHPINSEWQTDNCETCTCYET

SEQ ID NO: 75 - protein, 33 aa, Artificial
  PCK3145 derivative
DSTRKCMDLKGNKHPINSEWQTDNCETCTCYET

SEQ ID NO: 76 - protein, 34 aa, Artificial
  PCK3145 derivative
GDSTRKCMDLKGNKHPINSEWQTDNCETCTCYET

SEQ ID NO: 77 - protein, 35 aa, Artificial
  PCK3145 derivative
PGDSTRKCMDLKGNKHPINSEWQTDNCETCTCYET

SEQ ID NO: 78 - protein, 36 aa, Artificial
  PCK3145 derivative
VPGDSTRKCMDLKGNKHPINSEWQTDNCETCTCYET

SEQ ID NO: 79 - protein, 37 aa, Artificial
  PCK3145 derivative
GVPGDSTRKCMDLKGNKHPINSEWQTDNCETCTCYET

SEQ ID NO: 80 - protein, 38 aa, Artificial
  PCK3145 derivative
EGVPGDSTRKCMDLKGNKHPINSEWQTDNCETCTCYET

SEQ ID NO: 81 - protein, 39 aa, Artificial
  PCK3145 derivative
NEGVPGDSTRKCMDLKGNKHPINSEWQTDNCETCTCYET

SEQ ID NO: 82 - protein, 40 aa, Artificial
  PCK3145 derivative
PNEGVPGDSTRKCMDLKGNKHPINSEWQTDNCETCTCYET

SEQ ID NO: 83 - protein, 41 aa, Artificial
  PCK3145 derivative
IPNEGVPGDSTRKCMDLKGNKHPINSEWQTDNCETCTCYET

SEQ ID NO: 84 - protein, 42 aa, Artificial
  PCK3145 derivative
FIPNEGVPGDSTRKCMDLKGNKHPINSEWQTDNCETCTCYET

SEQ ID NO: 85 - protein, 43 aa, Artificial
  PCK3145 derivative
YFIPNEGVPGDSTRKCMDLKGNKHPINSEWQTDNCETCTCYET

SEQ ID NO: 86 - protein, 44 aa, Artificial
  PCK3145 derivative
CYFIPNEGVPGDSTRKCMDLKGNKHPINSEWQTDNCETCTCYET

SEQ ID NO: 87 - protein, 45 aa, Artificial
  PCK3145 derivative
SCYFIPNEGVPGDSTRKCMDLKGNKHPINSEWQTDNCETCTCYET

SEQ ID NO: 88 - protein, 15 aa, Artificial
  PCK3145 analog
XWQXDXCXXCXCXXX

SEQ ID NO: 89 - protein, 30 aa, Artificial
  PCK3145 derivative
EWQTDNCETCTCYETEWQTDNCETCTCYET

SEQ ID NO: 90 - protein, 45 aa, Artificial
  PCK3145 derivative
EWQTDNCETCTCYETEWQTDNCETCTCYETEWQTDNCETCTCYET

SEQ ID NO: 91 - protein, 60 aa, Artificial
  PCK3145 derivative
EWQTDNCETCTCYETEWQTDNCETCTCYETEWQTDNCETCTCYETEWQTDNCETCTCYET

SEQ ID NO: 92 - protein, 15 aa, Artificial
  PCK3145 analog
EYQTDNCETCTCYET

SEQ ID NO: 93 - protein, 15 aa, Artificial
  PCK3145 analog
EWNTDNCETCTCYET

SEQ ID NO: 94 - protein, 15 aa, Artificial
  PCK3145 analog
EWQTDQSETCTCYDT

SEQ ID NO: 95 - protein, 14 aa, Artificial
  PCK3145 fragment
WQTDNCETCTCYET

SEQ ID NO: 96 - protein, 13 aa, Artificial
  PCK3145 fragment
QTDNCETCTCYET

SEQ ID NO: 97 - protein, 12 aa, Artificial
  PCK3145 fragment
EWQTDNCETCTC

SEQ ID NO: 98 - protein, 15 aa, Artificial
  PCK3145 derivative
EWQTDNCETCTCXET

SEQ ID NO: 99 - protein, 15 aa, Artificial
  scrambled peptide
TCENCTETQWCETDY

(19) United States
(12) Patent Application Publication
(10) Pub. No.: US 2005/0148514 A1
(43) Pub. Date: Jul. 7, 2005

(54) Method and composition for treatment of angiogenesis

(76) Inventors: Chandra J. Panchal, London (CA); Jinzi Jason Wu, Dollard-des-Ormeaux (CA); Richard Beliveau, Montreal (CA); Marcia Ruiz, Ste-Genevieve (CA); Seema Garde, Montreal (CA); Borhane Annabi, Brossard (CA); Sylvie Lamy, Montreal (CA); Mounia Bouzeghrane, Montreal (CA); Luc Daigneault, Laval (CA); Robert Hawkins, Cheshire (GB)

Correspondence Address: TESTA, HURWITZ & THIBEAULT, LLP, HIGH STREET TOWER, 125 HIGH STREET, BOSTON, MA, 02110, US

(21) Appl. No.: 11/004,273

(22) Filed: Dec. 2, 2004

Related U.S. Application Data
(63) Continuation-in-part of application No. 10/948,229, filed on Sep. 24, 2004.

(30) Foreign Application Priority Data
Sep. 26, 2003 (CA) CA 2,441,695

(51) Int. Cl. A61K 38/17; A61K 38/52

(52) U.S. Cl. 514/12

(57) ABSTRACT

Angiogenesis, the formation of new blood vessels, is an integral part of normal physiological and developmental processes as well as several pathologies, ranging from tumor growth and metastasis to inflammation and ocular disease. Methods and compositions are provided for controlling normal angiogenesis and for treating angiogenesis associated or mediated diseases.

This application is a continuation-in-part and claims benefit of priority of U.S. patent application Ser. No. 10/948,229 filed on Sep. 24, 2004 and the benefit of priority of Canadian patent application no. 2,441,695 filed on Sep. 26, 2003, the entire content of which are incorporated herein by reference.

### FIELD OF THE INVENTION

The present invention relates to methods and compositions for treating or preventing unwanted angiogenesis in a human or animal. More particularly, the present invention relates to a method for treating or preventing unwanted angiogenesis, such as angiogenesis dependent or associated diseases, by administration of compounds such as PSP94 family members and related compounds.

### BACKGROUND OF THE INVENTION

The present invention relates to methods and compositions for effectively inhibiting angiogenesis. More specifically, the invention comprises PSP94 family members and their use in the inhibition of angiogenesis and treatment of angiogenesis associate diseases.

Angiogenesis refers to the formation of blood vessels into a tissue or organ. Under normal physiological conditions, humans or animals only undergo angiogenesis in very specific restricted situations. For example, angiogenesis is normally observed in wound healing, fetal and embryonal development and formation of the corpus luteum, endometrium and placenta. The control of angiogenesis is a highly regulated system of angiogenic stimulators and inhibitors. The control of angiogenesis has been found to be altered in certain disease states and, in many cases, the pathological damage associated with the disease is related to the uncontrolled angiogenesis.

Both controlled and uncontrolled angiogenesis are thought to proceed in a similar manner. Endothelial cells and pericytes which are surrounded by a basement membrane form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel. In the disease state, prevention of angiogenesis could avert the damage caused by the invasion of the new microvascular system.

Maturation and stabilization of the newly formed blood vessel occur via recruitment of pericytes and involve principally but not exclusively platelet-derived growth factor (PDGF), fibroblast growth factor-2 (FGF-2), transforming growth factor-beta (TGF-beta), vascular endothelial growth factor (VEGF) and angiopoietins (Darland, D. C. and P. A. D'Amore (1999) Journal of Clinical Investigation, 103: 157-58). A number of angiogenic factors have been identified based on the ability to promote the development of new blood vessels in vivo.

Persistent, unregulated angiogenesis occurs in a multiplicity of disease states. The diverse pathological states created due to unregulated angiogenesis have been grouped together as angiogenic dependent or angiogenic associated diseases. Therapies directed at control of the angiogenic processes could lead to the abrogation or mitigation of these diseases.

One example of a disease mediated by angiogenesis is ocular neovascular disease. This disease is characterized by invasion of new blood vessels into the structures of the eye such as the retina or cornea. It is the most common cause of blindness and is involved in approximately twenty eye diseases. In age-related macular degeneration, the associated visual problems are caused by an ingrowth of chorioidal capillaries through defects in Bruch's membrane with proliferation of fibrovascular tissue beneath the retinal pigment epithelium. Angiogenic damage is also associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Kaposi's sarcoma, Mooren's ulcer, Terrien's marginal degeneration, mariginal keratolysis, rheumatoid arthritis, systemic lupus, polyarteritis, trauma, Wegener's sarcoidosis, scleritis, Stevens-Johnson disease, pemphigoid, radial keratotomy, and corneal graph rejection.

Diseases associated with retinal/choroidal neovascularization include, but are not limited to, diabetic retinopathy, macular degeneration, sickle cell anemia, sarcoid, syphilis, pseudoxanthoma elasticum, Paget's disease, vein occlusion, artery occlusion, carotid obstructive disease, chronic uveitis/vitritis, mycobacterial infections, Lyme's disease, systemic lupus erythematosis, retinopathy of prematurity, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovasculariation of the angle) and diseases caused by the abnormal proliferation of fibrovascular or fibrous tissue including all forms of proliferative vitreoretinopathy.

Another disease in which angiogenesis is believed to be involved is rheumatoid arthritis. The blood vessels in the synovial lining of the joints undergo angiogenesis. In addition to forming new vascular networks, the endothelial cells release factors and reactive oxygen species that lead to pannus growth and cartilage destruction. The factors involved in angiogenesis may actively contribute to, and help maintain, the chronically inflamed state of rheumatoid arthritis.

Factors associated with angiogenesis may also have a role in osteoarthritis. The activation of the chondrocytes by angiogenic-related factors contributes to the destruction of the joint. At a later stage, the angiogenic factors would promote new bone formation. Therapeutic intervention that prevents the bone destruction could halt the progress of the disease and provide relief for persons suffering with arthritis.

Chronic inflammation may also involve pathological angiogenesis. Such disease states as ulcerative colitis and Crohn's disease show histological changes with the ingrowth of new blood vessels into the inflamed tissues. Bartonellosis, a bacterial infection found in South America, can result in a chronic stage that is characterized by proliferation of vascular endothelial cells. Another pathological role associated with angiogenesis is found in atherosclerosis. The plaques formed within the lumen of blood vessels have been shown to have angiogenic stimulatory activity.

Angiogenesis is also responsible for damage found in hereditary diseases such as Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula.

Angiogenesis is prominent in solid tumor formation and metastasis. Angiogenic factors have been found associated with several solid tumors such as rhabdomyosarcomas, retinoblastoma, Ewing sarcoma, neuroblastoma, and osteosarcoma. A tumor cannot expand without a blood supply to provide nutrients and remove cellular wastes. Tumors in which angiogenesis is important include solid tumors, and benign tumors such as acoustic neuroma, neurofibroma, trachoma and pyogenic granulomas. Prevention of angiogenesis could halt the growth of these tumors and the resultant damage to the animal due to the presence of the tumor.

It should be noted that angiogenesis has been associated with blood-born tumors such as leukemias, any of various acute or chronic neoplastic diseases of the bone marrow in which unrestrained proliferation of white blood cells occurs, usually accompanied by anemia, impaired blood clotting, and enlargement of the lymph nodes, liver, and spleen. It is believed that angiogenesis plays a role in the abnormalities in the bone marrow that give rise to leukemia-like tumors.

Angiogenesis is important in two stages of tumor metastasis. The first stage where angiogenesis stimulation is important is in the vascularization of the tumor which allows tumor cells to enter the blood stream and to circulate throughout the body. After the tumor cells have left the primary site, and have settled into the secondary, metastasis site, angiogenesis must occur before the new tumor can grow and expand. Therefore, prevention of angiogenesis could lead to the prevention of metastasis of tumors and possibly contain the neoplastic growth at the primary site.

Knowledge of the role of angiogenesis in the maintenance and metastasis of tumors has led to a prognostic indicator for breast cancer. The amount of neovascularization found in the primary tumor was determined by counting the microvessel density in the area of the most intense neovascularization in invasive breast carcinoma. A high level of microvessel density was found to correlate with tumor recurrence. Control of angiogenesis by therapeutic means could possibly lead to cessation of the recurrence of the tumors.

Angiogenesis is also involved in normal physiological processes such as reproduction and wound healing. Angiogenesis is an important step in ovulation and also in implantation of the blastula after fertilization. Prevention of angiogenesis could be used to induce amenorrhea, to block ovulation or to prevent implantation by the blastula.

In wound healing, excessive repair or fibroplasia can be a detrimental side effect of surgical procedures and may be caused or exacerbated by angiogenesis. Adhesions are a frequent complication of surgery and lead to problems such as small bowel obstruction.

Matrix metalloproteinases (MMPs) play an important role in morphogenesis, angiogenesis, wound healing, and in certain disorders such as rheumatoid arthritis, tumor invasion and metastasis (Birkedal-Hansen, 1995, Curr. Opin. Cell Biol. 7: 728-735). MMPs are involved, for example, in physiological function where rearrangements of basement membranes occur.

Prostate secretory protein (PSP94) constitutes one of the three predominant proteins found in human seminal fluid along with prostate specific antigen (PSA) and prostatic acid phosphatase (PAP). PSP94 has a molecular weight of 10.7 kDa and contains 10 cysteine residues. The cDNA and the gene coding for PSP94 have been cloned and characterized.

It was shown that PSP94 inhibits growth of tumor cells (see U.S. Pat. No. 5,428,011 to Seth et al., the entire content of which is incorporated herein by reference). Tumor growth inhibition by PSP94 fragment, has also been observed in animal models (see International application No. PCT/CA01/01463 to Garde, S. et al., published under No.: WO02/33090, the entire content of which is incorporated herein by reference). PSP94 also reduces the development of skeletal metastasis (see International application No.: PCT/CA02/01737 to Rabbani, S. et al., published under No.: WO03/039576, the entire content of which is incorporated herein by reference). This latter characteristic was observed by a reduction in calcium levels following administration of PSP94 to animal modeling prostate cancer.

### SUMMARY OF THE INVENTION

The present invention relates to the reduction or inhibition of angiogenesis by a PSP94 family member.

In accordance with the present invention a PSP94 family member is used in angiogenesis mediated disease, in angiogenesis associated disease or in the inhibition of normal angiogenesis.

In accordance with the present invention, compositions and methods are provided that are effective in inhibiting, for example, unwanted angiogenesis. The present invention provides a method of treating or preventing diseases (e.g., in a mammal in need) mediated, for example, by undesired or uncontrolled angiogenesis by administering a composition comprising an anti-angiogenic compound (a PSP94 family member) in a dosage sufficient to inhibit angiogenesis.

In accordance with the present invention, angiogenesis-mediated or associated diseases encompassed by the present invention comprise for example, ocular neovascularization (e.g., cornea, retina), macular degeneration, cancer-associated angiogenesis, metastasis-associated angiogenesis, retrolental fibroplasia, psoriasis, diabetic retinopathy, retrolental fibroplasia, Crohn's disease, etc.

Therefore, PSP94, PSP94 derivatives, PCK3145, PCK3145 derivatives, fragments, analogues and homologues thereof may therefore find utility in cancer treatment, wound healing, anti-angiogenesis, anti-inflammation, anti-osteoarthritis, inhibition of hair growth, reduction of degradation of some cytokine (e.g., IFN-beta) as well as for skin treatment (e.g., prevention of blistering photo-aging, psoriasis), wound healing, tissue remodeling, pulmonary fibrosis, etc.

In accordance with the present invention a PSP94 family member may be used to inhibit or to reduce normal angiogenesis, for example angiogenesis associated with the menstrual cycle of a woman (endometrial angiogenesis).

Aspects of the present invention relate to the use of a PSP94 family member (in an isolated cell, a cell lyzate, in a tissue, in an individual (a mammal) etc.) for 1) the inhibition or reduction of angiogenesis, 2) the inhibition or reduction of VEGF receptor phosphorylation or activation (e.g., reduction of the tyrosine kinase signal transduction activity), 3) the inhibition or reduction of PDGF receptor phosphorylation or activation (e.g., reduction of the tyrosine kinase signal transduction activity), 4) inhibition or reduction of the ability of a VEGF receptor to phosphorylate a substrate, 5) inhibition or reduction of the ability of a PDGF receptor to phosphorylate a substrate, 6) inhibition or reduction of VEGF-mediated and/or VEGFR-mediated ERK phosphorylation 7) inhibition or reduction of PDGF-mediated and/or PDGFR-mediated ERK phosphorylation, 8) inhibition or reduction of constitutive secretion, 9) increase or stimulation of protein (RNA) expression from a gene having at least one Serum Response Element (SRE), 10) increase or stimulation of protein (RNA) expression from a gene having at least one NF-KB element; 11) increase or stimulation of ERK phosphorylation, 12) increase or stimulation of the MAPK/JNK pathway, 13) increase or stimulation of apoptosis and any combinations thereof.

In additional aspect, the present invention relates to the use of a PSP94 family member to treat a condition or disease associated with 1) angiogenesis, 2) VEGF receptor phosphorylation, 3) PDGF receptor phosphorylation, 4) phosphorylation of a substrate mediated by a VEGF receptor, 5) phosphorylation of a substrate mediated by a PDGF receptor, 6) VEGF-mediated and/or VEGFR-mediated ERK phosphorylation 7) PDGF-mediated and/or PDGFR-mediated ERK phosphorylation, 8) insufficient of poor protein (or RNA) expression from a gene having at least one Serum Response Element (SRE), 9) insufficient of poor protein (or RNA) expression from a gene having at least one NF-KB element; 10) insufficient of poor stimulation of ERK phosphorylation, 11) insufficient or poor stimulation of the MAPK/JNK pathway and any combinations thereof.

It may also be useful to use a PSP94 family member to treat a condition or disease associated with unwanted cell growth by promoting apoptosis in the cell.

More particularly, the present invention relates to the use of a compound which may be selected from the group consisting of SEQ ID NO.:5, a SEQ ID NO.:5 derivative able to reduce tube formation in an angiogenesis assay, a SEQ ID NO.:5 fragment able to reduce tube formation in an angiogenesis assay, a SEQ ID NO.:5 analog able to reduce tube formation in an angiogenesis assay and combination thereof in the treatment of angiogenesis in an individual in need.

Also, more particularly, the present invention relates to the use of a compound selected from the group consisting of SEQ ID NO.:5, a SEQ ID NO.:5 derivative able to reduce at least one of substrate phosphorylation by VEGF receptor, substrate phosphorylation by PDGF receptor and substrate phosphorylation by VEGF receptor and PDGF receptor, a SEQ ID NO.:5 fragment able to reduce at least one of substrate phosphorylation by VEGF receptor, substrate phosphorylation by PDGF receptor and substrate phosphorylation by VEGF receptor and PDGF receptor, a SEQ ID NO.:5 analog able to reduce at least one of substrate phosphorylation by VEGF receptor, substrate phosphorylation by PDGF receptor and substrate phosphorylation by VEGF receptor and PDGF receptor and combination thereof, in the treatment of a disease associated with phosphorylation of VEGF receptor, phosphorylation of PDGF receptor, phosphorylation of a substrate mediated by a VEGF receptor, phosphorylation of a substrate mediated by a PDGF receptor and/or combination thereof.

In an additional aspect, the present invention relates to a compound selected from the group consisting of, SEQ ID NO.:5, a SEQ ID NO.:5 derivative, a SEQ ID NO.:5 fragment, a SEQ ID NO.:5 analog and combination thereof, as well any PSP94 family member for treating a disease selected from diseases associated with (or mediated by); 1) angiogenesis, 2) VEGF receptor phosphorylation, 3) PDGF receptor phosphorylation, 4) phosphorylation of a substrate mediated by a VEGF receptor, 5) phosphorylation of a substrate mediated by a PDGF receptor, 6) VEGF-mediated and/or VEGFR-mediated ERK phosphorylation 7) PDGF-mediated and/or PDGFR-mediated ERK phosphorylation, 8) insufficient of poor protein (or RNA) expression from a gene having at least one Serum Response Element (SRE), 9) insufficient of poor protein (or RNA) expression from a gene having at least one NF-KB element; 10) insufficient of poor stimulation of ERK phosphorylation, 11) insufficient or poor stimulation of the MAPK/JNK pathway and any combinations thereof, the method may comprise administering a PSP94 family member (e.g., a drug or pharmaceutical composition comprising a PSP94 family member) to a patient in need thereof.

In yet an additional aspect, the present invention relates to a method (of treatment) and pharmaceutical compositions for treating a disease selected from diseases associated with (or mediated by); 1) angiogenesis, 2) VEGF receptor phosphorylation, 3) PDGF receptor phosphorylation, 4) phosphorylation of a substrate mediated by a VEGF receptor, 5) phosphorylation of a substrate mediated by a PDGF receptor, 6) VEGF-mediated and/or VEGFR-mediated ERK phosphorylation 7) PDGF-mediated and/or PDGFR-mediated ERK phosphorylation, 8) insufficient of poor protein (or RNA) expression from a gene having at least one Serum Response Element (SRE), 9) insufficient of poor protein (or RNA) expression from a gene having at least one NF-KB element; 10) insufficient of poor stimulation of ERK phosphorylation, 11) insufficient or poor stimulation of the MAPK/JNK pathway and any combinations thereof, the method may comprise administering a PSP94 family member (e.g., a drug or pharmaceutical composition comprising a PSP94 family member) to a patient in need thereof.

More particularly, the present invention provides a method of inhibiting angiogenesis in an individual in need thereof, the method may comprise, for example, administering to the individual a compound which may be selected from the group consisting of,
a) SEQ ID NO.:5,
b) a SEQ ID NO.:5 derivative able to reduce tube formation in an angiogenesis assay,
c) a SEQ ID NO.:5 fragment able to reduce tube formation in an angiogenesis assay,
d) a SEQ ID NO.:5 analog able to reduce tube formation in an angiogenesis assay, and;
e) combination of any one of a) through d) thereof.

In accordance with the present invention, the compound may further comprise a grouping for increasing the stability of the compound. Further in accordance with the present invention, the grouping may be, for example, an acetylaminomethyl moiety attached to a sulfur atom of a cysteine. The compound may be, for example, SEQ ID NO.:7.

Also in accordance with the present invention, the method may be used for treating cancer-associated angiogenesis or metastasis-associated angiogenesis.

In a further aspect, the present invention provides a pharmaceutical composition for treating a condition or a disease described herein, the pharmaceutical composition may comprise;
a PSP94 family member, and;
a pharmaceutically acceptable carrier.

More particularly, the present invention relates to a pharmaceutical composition for treating angiogenesis or an ocular disease, the composition may comprise, for example, a compound selected from the group consisting of,
SEQ ID NO.:5, a SEQ ID NO.:5 derivative able to reduce tube formation in an angiogenesis assay, a SEQ ID NO.:5 fragment able to reduce tube formation in an angiogenesis assay, a SEQ ID NO.:5 analog able to reduce tube formation in an angiogenesis assay and combination thereof, and;
a pharmaceutically acceptable carrier.

The present invention also relates to the use of a PSP94 family member in the manufacture of a pharmaceutical composition or a drug for the treatment of one or more disease or condition described herein.

More particularly, the present invention relates to the use of a compound which may be selected, for example, from the group consisting of SEQ ID NO.:5, a SEQ ID NO.:5 derivative able to reduce tube formation in an angiogenesis assay, a SEQ ID NO.:5 fragment able to reduce tube formation in an angiogenesis assay, a SEQ ID NO.:5 analog able to reduce tube formation in an angiogenesis assay and combination thereof in the manufacture of a pharmaceutical composition for the treatment of angiogenesis.

Also more particularly the present invention relates to the use of a compound, which may be selected from the group consisting of SEQ ID NO.:5, a SEQ ID NO.:5 derivative able to reduce at least one of substrate phosphorylation by VEGF receptor, substrate phosphorylation by PDGF receptor and substrate phosphorylation by VEGF receptor and PDGF receptor, a SEQ ID NO.:5 fragment able to reduce at least one of substrate phosphorylation by VEGF receptor, substrate phosphorylation by PDGF receptor and substrate phosphorylation by VEGF receptor and PDGF receptor, a SEQ ID NO.:5 analog able to reduce at least one of substrate phosphorylation by VEGF receptor, substrate phosphorylation by PDGF receptor and substrate phosphorylation by VEGF receptor and PDGF receptor and combination thereof, in manufacture of a pharmaceutical composition for the treatment of a disease associated with phosphorylation of VEGF receptor, phosphorylation of PDGF receptor, phosphorylation of a substrate mediated by a VEGF receptor, phosphorylation of a substrate mediated by a PDGF receptor and combination thereof.

An example of a disease which may be treated by blocking phosphorylation by VEGF and PDGF receptors is ocular neovascularization (retina) (Ozaki, H., et al., Am. J. Pathol., 156 (2): 697-707, 2000; the entire content of which is incorporated herein by reference).

The present invention, in a further aspect thereof, relates to a method of treating a disease associated with substrate-phosphorylation by VEGF receptor, substrate-phosphorylation by PDGF receptor or substrate-phosphorylation by both VEGF and PDGF receptors, the method may comprise administering a compound (in a therapeutically effective amount) which is a PSP94 family member to an individual in need thereof.

In accordance with the present invention, the substrate may be a kinase such as Extracellular-signal-Regulated protein Kinases (ERK).

Therefore, the present invention also relates to a method of treating retinal vascularization by inhibiting VEGF receptor tyrosine kinase signal transduction, PDGF receptor tyrosine kinase signal transduction or both VEGF receptor tyrosine kinase signal transduction and PDGF receptor tyrosine kinase signal transduction, which method comprises administering to a individual in need of such treatment a therapeutically effective amount of a PSP94 family member.

More particularly, the present invention provides a method of treating a disease associated with phosphorylation of VEGF receptor, b) phosphorylation of PDGF receptor, c) phosphorylation of a substrate mediated by a VEGF receptor, d) phosphorylation of a substrate mediated by a PDGF receptor and combination of any of a) through d) thereof, the method may comprise administering to an individual in need thereof, a compound which may be selected from the group consisting of SEQ ID NO.:5, a SEQ ID NO.:5 derivative able to reduce at least one of substrate phosphorylation by VEGF receptor, substrate phosphorylation by PDGF receptor and substrate phosphorylation by VEGF receptor and PDGF receptor, a SEQ ID NO.:5 fragment able to reduce at least one of substrate phosphorylation by VEGF receptor, substrate phosphorylation by PDGF receptor and substrate phosphorylation by VEGF receptor and PDGF receptor and a SEQ ID NO.:5 analog able to reduce at least one of substrate phosphorylation by VEGF receptor, substrate phosphorylation by PDGF receptor and substrate phosphorylation by VEGF receptor and PDGF receptor.

In accordance with the present invention, the disease may be an ocular disease, such as, for example, ocular neovascularization.

The invention further provides, in an additional aspect, a method of regulating, in a cell (in tissue culture (ex vivo), in a tissue, in a human (body), etc.), an abnormal activation of a molecule selected, for example, from the group of molecules (polypeptides) consisting of a VEGF receptor, a PDGF receptor, a downstream effector activated by a VEGF receptor and a downstream effector activated by a PDGF receptor, the method comprising contacting the cell with a PSP94 family member.

In accordance with the present invention, the abnormal activation of the VEGF receptor may be, for example, an increase in the tyrosine kinase signal transduction activity of the VEGF receptor. Further in accordance with the present invention, the abnormal activation of the VEGF receptor or the downstream effector activated by a VEGF receptor, may be promoted, for example by VEGF.

In accordance with the present invention, the abnormal activation of the PDGF receptor may be, for example, an increase in the tyrosine kinase signal transduction activity of the PDGF receptor. Further in accordance with the present invention, the abnormal activation of the PDGF receptor or the downstream effector activated by a PDGF receptor may be promoted, for example by PDGF.

Members of the PSP94 family (or PSP94 family member) comprises, for example, PSP94 (SEQ ID NO.:1), a PSP94 fragment, a PSP94 derivative, a PSP94 analogue, PCK3145 (SEQ ID NO.:5), a PCK3145 fragment, a PCK3145 derivative and a PCK3145 analogue. A PCK3145 derivative may be, for example, as defined in SEQ ID NO.:7. PSP94 family members therefore also include, for example, SEQ ID NO.:2, SEQ ID NO.: 3, SEQ ID NO.:4, SEQ ID NO.:6, as well as SEQ ID NO.: 9 to 98.

In accordance with the present invention, the member of the PSP94 family (PSP94 family member) may be selected, for example, from the group consisting of; a) SEQ ID NO.:1, b) a SEQ ID NO.:1 derivative, c) a SEQ ID NO.:1 fragment, d) SEQ ID NO.:1 analogue, e) SEQ ID NO.:5, f) a SEQ ID NO.:5 derivative, g) a SEQ ID NO.:5 fragment, h) a SEQ ID NO.:5 analogue, i) SEQ ID NO.:7, and j) combination of any one of a) through i) thereof.

It is to be understood herein that peptide derivatives, fragments and analogues of the present invention may be chosen among those which have a desired biological activity. Derivatives, fragments and analogues encompassed by the present invention are those which have one or more of the following biological activity, for examples, those which 1) inhibit or reduce angiogenesis, 2) inhibit or reduce tubulogenesis, 3) inhibit or reduce phosphorylation of VEGF receptor, 4) inhibit or reduce phosphorylation of PDGF receptor, 5) inhibit or reduce phosphorylation of a substrate mediated by a VEGF receptor, 6) inhibit or reduce phosphorylation of a substrate mediated by a PDGF receptor, 7) inhibit or reduce VEGF-mediated ERK phosphorylation, 8) inhibit or reduce PDGF-mediated ERK phosphorylation, 9) increase or stimulate expression from a gene having at least one Serum Response Element (SRE), 10) increase or stimulate expression from a gene having at least one NF-KB element, 11) increase or stimulate the MAPK/JNK pathway or 12) increase or stimulate apoptosis.

This invention also relates to the regulation (either directly or indirectly) of matrix metalloproteinases (MMPs), (e.g., MMP-9, MMP-2, MT1-MMP) by PSP94 family members. More particularly, the present invention relates to the use of a PSP94 family member for the treatment of a condition related to the activity or expression of MMPs or pro-MMPs.

In another aspect, the present invention provides a compound and the use of a compound which is a member of the PSP94 family in the treatment of a condition related, for example, to the activity or to the expression of a polypeptide which may be, for example, selected from the group consisting of matrix metalloproteinases and pro-matrix metalloproteinases.

In another aspect, the present invention provides a compound having the biological activity of PCK3145 (SEQ ID NO.:5) which may comprise or consist essentially of the amino acid sequence identified in SEQ ID NO.:5 and may further comprise a stabilizing group (e.g. a group increasing in vivo stability of the compound or polypeptide without affecting deleteriously the biological activity of the compound or polypeptide) covalently attached to an amino acid of the (SEQ ID NO.:5) sequence.

In accordance with the present invention the group may be, for example, an acetylaminomethyl group attached to a sulfur atom of a cysteine or a polyethylene glycol (PEG) group attached to at least one amino acid of the sequence or any other modification which improves a desired property (e.g., stability) of the compound/polypeptide.

As used herein, “VEGF” means vascular endothelial growth factor and VEGFR or VEGF-R means vascular endothelial growth factor receptor. Similarly, VEGFR-2 means endothelial growth factor receptor type-2.

As used herein, “PDGF” means platelet-derived growth factor and PDGFR or PDGF-R means platelet-derived growth factor receptor.

A “PSP94 family member” or “a member of the PSP94 family” is further understood herein as any polypeptide originating from PSP94. For example, “PSP94 family members” may comprise wild type PSP94 (SEQ ID NO.:1) a PSP94 fragment, a PSP94 derivative, a PSP94 analogue, PCK3145 (SEQ ID NO.:5), a PCK3145 fragment, a PCK3145 derivative, a PCK3145 analogue, etc.

A “fragment” is to be understood herein as a polypeptide originating from a portion of an original or parent sequence. Fragments encompass polypeptides having truncations of one or more amino acids, wherein the truncation may originate from the amino terminus (N-terminus), carboxy terminus (C-terminus), or from the interior of the protein. A fragment may comprise the same sequence as the corresponding portion of the original sequence. For example, SEQ ID NO.: 4, SEQ ID NO.: 5 and SEQ ID NO.: 6 fall into the definition of “a PSP94 fragment”; when considering PSP94 (SEQ ID NO.:1) as an original sequence.

A “derivative” is to be understood herein as a polypeptide originating from an original sequence or from a portion of an original sequence and which may comprise one or more modification; for example, one or more modification in the amino acid sequence (e.g., an amino acid addition, deletion, insertion, substitution etc.), one or more modification in the backbone or side-chain of one or more amino acid, or an addition of a group or another molecule to one or more amino acids (side-chains or backbone). For example, SEQ ID NO.: 2, SEQ ID NO.: 3 and SEQ ID NO.: 7 fall into the definition of “a PSP94 derivative”; when considering PSP94 (SEQ ID NO.:1) as an original sequence.

It is to be understood herein that SEQ ID NO.: 7 may fall into the definition of “a PCK3145 derivative” or “SEQ ID NO.:5 derivative) when considering PCK3145 (SEQ ID NO.:5) as an original sequence. The addition of polyethylene glycol group (i.e., pegylation) to PCK3145 (SEQ ID NO.:5 or SEQ ID NO.: 7) also falls within the definition of “a PCK3145 derivative”.

An “analogue” is to be understood herein as a molecule having a biological activity and chemical structure similar to that of a polypeptide described herein. An “analogue” may have sequence similarity with that of an original sequence or a portion of an original sequence and may also have a modification of its structure as discussed herein. For example, an “analogue” may have at least 90% sequence similarity with an original sequence or a portion of an original sequence. An “analogue” may also have, for example; at least 70% or even 50% sequence similarity (or less, i.e., at least 40%) with an original sequence or a portion of an original sequence. Also, an “analogue” may have, for example, 50% sequence similarity to an original sequence with a combination of one or more modification in a backbone or side-chain of an amino acid, or an addition of a group or another molecule, etc.

Thus, biologically active polypeptides in the form of the original polypeptides, fragments (modified or not), analogues (modified or not), derivatives (modified or not), homologues, (modified or not) of PSP94 and PCK3145 are encompassed by the present invention.

Therefore, any polypeptide having a modification compared to an original polypeptide (e.g., PSP94, PCK3145) which does not destroy significantly a desired biological activity is encompassed herein. It is well known in the art, that a number of modifications may be made to the polypeptides of the present invention without deleteriously affecting their biological activity. These modifications may, on the other hand, may keep or increase the biological activity of the original polypeptide or may optimize one or more of the particularity (e.g. stability, bioavailability, etc.) of the polypeptides of the present invention which, in some instance might be desirable. Polypeptides of the present invention comprises for example, those containing amino acid sequences modified either by natural processes, such as posttranslational processing, or by chemical modification techniques which are known in the art. Modifications may occur anywhere in a polypeptide including the polypeptide backbone, the amino acid side-chains and the amino- or carboxy-terminus. It will be appreciated that the same type of modification may be present in the same or varying degrees at several sites in a given polypeptide. Also, a given polypeptide may contain many types of modifications. Polypeptides may be branched as a result of ubiquitination, and they may be cyclic, with or without branching. Cyclic, branched and branched cyclic polypeptides may result from posttranslational natural processes or may be made by synthetic methods. Modifications comprise for example, without limitation, pegylation, acetylation, acylation, addition of acetomidomethyl (Acm) group, ADP-ribosylation, alkylation, amidation, biotinylation, carbamoylation, carboxyethylation, esterification, covalent attachment to flavin, covalent attachment to a heme moiety, covalent attachment of a nucleotide or nucleotide derivative, covalent attachment of drug, covalent attachment of a marker (e.g., fluorescent, radioactive, etc.), covalent attachment of a lipid or lipid derivative, covalent attachment of phosphatidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation and ubiquitination, etc. It is to be understood herein that more than one modification to the polypeptides described herein are encompassed by the present invention to the extent that the biological activity is similar to the original (parent) polypeptide.

As discussed above, polypeptide modification may comprise, for example, amino acid insertion (i.e., addition), deletion and substitution (i.e., replacement), either conservative or non-conservative (e.g., D-amino acids, desamino acids) in the polypeptide sequence where such changes do not substantially alter the overall biological activity of the polypeptide.

Example of substitutions may be those, which are conservative (i.e., wherein a residue is replaced by another of the same general type or group) or when wanted, non-conservative (i.e., wherein a residue is replaced by an amino acid of another type). In addition, a non-naturally occurring amino acid may substitute for a naturally occurring amino acid (i.e., non-naturally occurring conservative amino acid substitution or a non-naturally occurring non-conservative amino acid substitution).

As is understood, naturally occurring amino acids may be sub-classified as acidic, basic, neutral and polar, or neutral and non-polar. Furthermore, three of the encoded amino acids are aromatic. It may be of use that encoded polypeptides differing from the determined polypeptide of the present invention contain substituted codons for amino acids, which are from the same type or group as that of the amino acid be replaced. Thus, in some cases, the basic amino acids Lys, Arg and His may be interchangeable; the acidic amino acids Asp and Glu may be interchangeable; the neutral polar amino acids Ser, Thr, Cys, Gln, and Asn may be interchangeable; the non-polar aliphatic amino acids Gly, Ala, Val, Ile, and Leu are interchangeable but because of size Gly and Ala are more closely related and Val, Ile and Leu are more closely related to each other, and the aromatic amino acids Phe, Trp and Tyr may be interchangeable.

It should be further noted that if the polypeptides are made synthetically, substitutions by amino acids, which are not naturally encoded by DNA (non-naturally occurring or unnatural amino acid) may also be made.

A non-naturally occurring amino acid is to be understood herein as an amino acid which is not naturally produced or found in a mammal. A non-naturally occurring amino acid comprises a D-amino acid, an amino acid having an acetylaminomethyl group attached to a sulfur atom of a cysteine, a pegylated amino acid, etc. The inclusion of a non-naturally occurring amino acid in a defined polypeptide sequence will therefore generate a derivative of the original polypeptide. Non-naturally occurring amino acids (residues) include also the omega amino acids of the formula NH₂(CH₂)ₙCOOH wherein n is 2-6, neutral nonpolar amino acids, such as sarcosine, t-butyl alanine, t-butyl glycine, N-methyl isoleucine, norleucine, etc. Phenylglycine may substitute for Trp, Tyr or Phe; citrulline and methionine sulfoxide are neutral nonpolar, cysteic acid is acidic, and ornithine is basic. Proline may be substituted with hydroxyproline and retain the conformation conferring properties.

It is known in the art that analogues may be generated by substitutional mutagenesis and retain the biological activity of the polypeptides of the present invention. These analogues have at least one amino acid residue in the protein molecule removed and a different residue inserted in its place. For example, one site of interest for substitutional mutagenesis may include but are not restricted to sites identified as the active site(s), or immunological site(s). Other sites of interest may be those, for example, in which particular residues obtained from various species are identical. These positions may be important for biological activity. Examples of substitutions identified as “conservative substitutions” are shown in table 1. If such substitutions result in a change not desired, then other type of substitutions, denominated “exemplary substitutions” in table 1, or as further described herein in reference to amino acid classes, are introduced and the products screened.

In some cases it may be of interest to modify the biological activity of a polypeptide by amino acid substitution, insertion, or deletion. For example, modification of a polypeptide may result in an increase in the polypeptide's biological activity, may modulate its toxicity, may result in changes in bioavailability or in stability, or may modulate its immunological activity or immunological identity. Substantial modifications in function or immunological identity are accomplished by selecting substitutions that differ significantly in their effect on maintaining (a) the structure of the polypeptide backbone in the area of the substitution, for example, as a sheet or helical conformation. (b) the charge or hydrophobicity of the molecule at the target site, or (c) the bulk of the side chain. Naturally occurring residues are divided into groups based on common side chain properties:
(1) hydrophobic: norleucine, methionine (Met), Alanine (Ala), Valine (Val), Leucine (Leu), Isoleucine (Ile)
(2) neutral hydrophilic: Cysteine (Cys), Serine (Ser), Threonine (Thr)
(3) acidic: Aspartic acid (Asp), Glutamic acid (Glu)
(4) basic: Asparagine (Asn), Glutamine (Gln), Histidine (His), Lysine (Lys), Arginine (Arg)
(5) residues that influence chain orientation: Glycine (Gly), Proline (Pro); and aromatic: Tryptophan (Trp), Tyrosine (Tyr), Phenylalanine (Phe)

Non-conservative substitutions will entail exchanging a member of one of these classes for another.

TABLE 1
amino acid substitution

| Original residue | Exemplary substitution | Conservative substitution |
|---|---|---|
| Ala (A) | Val, Leu, Ile | Val |
| Arg (R) | Lys, Gln, Asn | Lys |
| Asn (N) | Gln, His, Lys, Arg | Gln |
| Asp (D) | Glu | Glu |
| Cys (C) | Ser | Ser |
| Gln (Q) | Asn | Asn |
| Glu (E) | Asp | Asp |
| Gly (G) | Pro | Pro |
| His (H) | Asn, Gln, Lys, Arg | Arg |
| Ile (I) | Leu, Val, Met, Ala, Phe, | Leu |
| | norleucine | |
| Leu (L) | Norleucine, Ile, Val, Met, | Ile |
| | Ala, Phe | |
| Lys (K) | Arg, Gln, Asn | Arg |
| Met (M) | Leu, Phe, Ile | Leu |
| Phe (F) | Leu, Val, Ile, Ala | Leu |
| Pro (P) | Gly | Gly |
| Ser (S) | Thr | Thr |
| Thr (T) | Ser | Ser |
| Trp (W) | Tyr | Tyr |
| Tyr (Y) | Trp, Phe, Thr, Ser | Phe |
| Val (V) | Ile, Leu, Met, Phe, Ala, | Leu |
| | norleucine | |

Example of biologically active analogues of PCK3145 (SEQ ID NO: 5) exemplified by amino acid substitutions is illustrated below.

| | | | | |
|---|---|---|---|---|
| Position | 1 | 5 | 10 | 15 |

| | | | | | | | | | | | | | | | |
|---|---|---|---|---|---|---|---|---|---|---|---|---|---|---|---|
| PCK3145 | E | W | Q | T | D | N | C | E | T | C | T | C | Y | E | T |
| (SEQ ID | X₁ | W | Q | X₂ | D | X₁ | C | X₁ | X₂ | C | X₂ | C | X₃ | X₁ | X₂ |
| NO.:88) | | | | | | | | | | | | | | | |

For example, X₁ may be glutamic acid (i.e., glutamate) (Glu), aspartic acid (aspartate) (Asp), or asparagine (Asn), X₂ may be threonine (Thr) or serine (Ser) and X₃ may be tyrosine (Tyr) or phenylalanine (Phe). Any replacement of an original residue in SEQ ID NO.:5 with a conserved amino acid (i.e. conservative substitution) is encompassed by the present invention.

Another example of a PCK3145 (SEQ ID NO: 5) analogue may include, for example, a polypeptide as exemplified in SEQ ID NO.:88 or any other polypeptide having at least one conservative amino acid substitution (illustrated in bold below) as defined in Table 1, such as, for example;

| | |
|---|---|
| Glu Tyr Gln Thr Asp Asn Cys Glu | (SEQ ID NO.:92) |
| Thr Cys Thr Cys Tyr Glu Thr | |
| Glu Trp Asn Thr Asp Asn Cys Glu | (SEQ ID NO.:93) |
| Thr Cys Thr Cys Tyr Glu Thr | |
| Glu Trp Gln Thr Asp Gln Ser Glu | (SEQ ID NO.:94) |
| Thr Cys Thr Cys Tyr Asp Thr | |

Examples of a PCK3145 (SEQ ID NO: 5) derivative may include, for example, a polypeptide having an addition in one or both of the terminal region (amino-terminal or carboxy-terminal) as illustrated in SEQ IDs No.: 9 to 87, or a peptide having a stabilizing group such as exemplified in SEQ ID NO.:7, or a peptide having one or more repeats of SEQ ID No.:5 such as exemplified in SEQ ID NOs.: 89 to 91, a polypeptide having at least one D-amino acid as exemplified in SEQ ID No. 98 and combination thereof.

An example of a PCK3145 (SEQ ID NO: 5) fragment may include, for example, a polypeptide having a truncation in one or both of the amino acid terminal region as illustrated below.

| | |
|---|---|
| Trp Gln Thr Asp Asn Cys Glu Thr | (SEQ ID NO.:95) |
| Cys Thr Cys Tyr Glu Thr | |
| Gln Thr Asp Asn Cys Glu Thr Cys | (SEQ ID NO.:96) |
| Thr Cys Tyr Glu Thr | |
| Glu Trp Gln Thr Asp Asn Cys Glu | (SEQ ID NO.:97) |
| Thr Cys Thr Cys | |

Polypeptides may be either naturally occurring (that is to say, substantially purified or isolated from a natural source) or synthetic (for example, by performing site-directed mutagenesis on the encoding DNA or made by other synthetic methods such as chemical synthesis). It is thus apparent that the polypeptides of the invention can be either naturally occurring or recombinant (that is to say prepared from the recombinant DNA techniques) or made by chemical synthesis (e.g., organic synthesis).

As used herein, “pharmaceutical composition” means therapeutically effective amounts of the agent together with pharmaceutically acceptable diluents, preservatives, solubilizers, emulsifiers, adjuvant and/or carriers. A “therapeutically effective amount” as used herein refers to that amount which provides a therapeutic effect for a given condition and administration regimen. Such compositions are liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bilo acid salts). Solubilizing agents (e.g., glycerol, polyethylene glycerol), anti-oxidants (e.g., ascorbic acid, sodium metabisulfite), preservatives (e.g., thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polyglycolic acid, hydrogels, etc, or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms protective coatings, protease inhibitors or permeation enhancers for various routes of administration, including parenteral, pulmonary, nasal, oral, vaginal, rectal routes. In one embodiment the pharmaceutical composition is administered parenterally, paracancerally, transmucosally, transdermally, intramuscularly, intravenously, intradermally, subcutaneously, intraperitonealy, intraventricularly, intracranially and intratumorally.

The formulations include those suitable for oral, rectal, ophthalmic, (including intravitreal or intracameral) nasal, topical (including buccal and sublingual), vaginal or parenteral (including subcutaneous, intramuscular, intravenous, intradermal, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface active or dispersing agent. Molded tablets may be made by molding, in a suitable machine, a mixture of the powdered compound moistened with an inert liquid diluent. The tablets may be optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. An example of a topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tamports, creams, gels, pastes, foams or spray formulations containing in addition to the active ingredient such carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tablets of the kind previously described.

Further, as used herein “pharmaceutically acceptable carrier” or “pharmaceutical carrier” are known in the art and include, but are not limited to, 0.01-0.1 M or 0.05 M phosphate buffer or 0.8% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like.

It is to be understood herein, that if a “range” or “group” of substances (e.g. amino acids), substituents” or the like is mentioned or if other types of a particular characteristic (e.g. temperature, pressure, chemical structure, time, etc.) is mentioned, the present invention relates to and explicitly incorporates herein each and every specific member and combination of sub-ranges or sub-groups therein whatsoever. Thus, any specified range or group is to be understood as a shorthand way of referring to each and every member of a range or group individually as well as each and every possible sub-ranges or sub-groups encompassed therein; and similarly with respect to any sub-ranges or sub-groups therein. Thus, for example,
with respect to a temperature greater than 100° C., this is to be understood as specifically incorporating herein each and every individual temperature state, as well as sub-range, above 100° C., such as for example 101° C., 105° C. and up, 110° C. and up, 115° C. and up, 110 to 135° C., 115° C. to 135° C., 102° C. to 150° C., up to 210° C., etc.;
   and similarly with respect to other parameters such as, concentrations, elements, etc. . . .

It is in particular to be understood herein that the polypeptides of the present invention each include each and every individual polypeptide described thereby as well as each and every possible mutant, variant, homolog, analogue or else whether such mutant, variant, homolog, analogue or else is defined as positively including particular polypeptides, as excluding particular polypeptides or a combination thereof; for example an exclusionary definition for a polypeptide analogue (e.g. X₁WQX₂DX₁CX₁X₂CX₂CX₃X₁X₂ (SEQ ID NO. 88)) may read as follows: “provided that when one of X₁ is glutamic acid and X₂ is threonine X₃ may not be phenylalanine”.

It is also to be understood herein that “g” or “gm” is a reference to the gram weight unit; that “C” is a reference to the Celsius temperature unit.

### BRIEF DESCRIPTION OF DRAWINGS

In drawings which illustrates exemplary embodiment of the present invention;

FIG. 1 is a picture of a zymography gel showing the effect of the PCK3145 derivative (SEQ ID NO.:7) on MMP-9 levels and activity on collagen type 1—treated MatLyLu cells (first lane:marker; second lane:cells; third lane:cells and collagen; fourth lane:cells, collagen and 500 μg/ml of SEQ ID NO.:7; fifth lane:cells, collagen and 1 mg/ml of SEQ ID NO.:7),

FIG. 2 is a picture of a western blot membrane showing the effect of the PCK3145 derivative (SEQ ID NO.:7) on MMP-9 expression level (fist lane: MMP9 standard; second lane:cells; third lane:cells and collagen; fourth lane:cells, collagen and 100 μg/ml of SEQ ID NO.:7; fifth lane: cells, collagen and 500 μg/ml of SEQ ID NO.:7; sixth lane: cells, collagen and 1 mg/ml of SEQ ID NO.:7),

FIG. 3 is a picture of a zymography gel showing the effect of the PCK3145 derivative (SEQ ID NO.:7) on MMP-2 levels and VEGF-induced MMP-2 levels and an histogram expressing the results in a quantitative manner (PD=PD98059, PCK=PCK3145 derivative),

FIG. 4A is picture of a western blot showing the effect of the PCK3145 derivative (SEQ ID NO.:7) on induced ERK phosphorylation and an histogram expressing the results in a quantitative manner (Ctl=control, PCK=PCK3145 derivative),

FIG. 4B is picture of a western blot showing the dose-dependent effect of the PCK3145 derivative (SEQ ID NO.:7) on VEGF-induced ERK phosphorylation and an histogram expressing the results in a quantitative manner (PD=PD98059, PCK=PCK3145 derivative),

FIG. 4C is picture of a western blot showing the absence of inhibition of VEGF-induced ERK phosphorylation by a scrambled polypeptide and an histogram expressing the results in a quantitative manner (SEQ ID NO.:99), (Ctl=control),

FIG. 4D is picture of a western blot of a time-course illustrating the reduction of VEGF-induced ERK phosphorylation by PCK3145 derivative (SEQ ID NO.:7) and an histogram expressing the results in a quantitative manner (Ctl=control, PCK=PCK3145 derivative),

FIG. 5 is a picture illustrating the effect of the PCK3145 derivative on capillary-like structure formation and an histogram expressing the results in a quantitative manner (PCK=PCK3145 derivative),

FIG. 6A is picture of a western blot showing the effect of the PCK3145 derivative (SEQ ID NO.:7) on VEGF-induced VEGFR-2 phosphorylation and an histogram expressing the results in a quantitative manner (Ctl=control, PCK=PCK3145 derivative),

FIG. 6B is a picture of a western blot showing the dose-dependent effect of the PCK3145 derivative (SEQ ID NO.:7) on VEGF-induced VEGFR-2 phosphorylation and an histogram expressing the results in a quantitative manner (PD=PD98059, PCK=PCK3145 derivative, PTK=PTK787),

FIG. 6C is a picture of a western blot showing the absence of inhibition of VEGF-induced VEGFR-2 phosphorylation by a scrambled polypeptide (SEQ ID NO.:99) and an histogram expressing the results in a quantitative manner, (Ctl=control),

FIG. 7A is a picture of a western blot showing the effect of the PCK3145 derivative (SEQ ID NO.:7) on PDGF-induced PDGFR phosphorylation and an histogram expressing the results in a quantitative manner (Ctl=control, PCK=PCK3145 derivative),

FIG. 7B is a picture of a western blot showing the dose-dependent effect of the PCK3145 derivative (SEQ ID NO.:7) on PDGF-induced ERK phosphorylation and an histogram expressing the results in a quantitative manner (PD=PD98059, PCK=PCK3145 derivative),

FIG. 8A is an histogram illustrating the results of alcaline phosphatase secretion in the presence or absence of the PCK3145 derivative (SEQ ID NO.:7) from cells containing a vector expressing a SEAP gene driven by (operatively linked with) specific response elements,

FIG. 8B is a picture of a western blot of a time-course assay illustrating the effect of PCK3145 derivative (SEQ ID NO.:7) on ERK phosphorylation (PCK=PCK3145 derivative),

FIG. 8C is a picture of a western blot of a dose-response assay illustrating the effect of PCK3145 derivative (SEQ ID NO.:7) on ERK phosphorylation (PCK=PCK3145 derivative), and;

FIG. 8D is a picture of a western blot illustrating the effect of PCK3145 derivative (SEQ ID NO.:7) on ERK phosphorylation (PCK=PCK3145 derivative).

### DETAILED DESCRIPTION OF THE INVENTION

Polypeptides which are members of the PSP94 family include; wild type PSP94 as defined in SEQ ID NO.: 1, a recombinant PSP94 as defined in SEQ ID NO.:2 and PSP94 derivatives, fragments and analogues as defined, for example in the amino acid sequence defined in SEQ ID NO.: 3, SEQ ID NO.:4, SEQ ID NO.:5, SEQ ID NO.:6 and SEQ ID NO.:7.

PCK3145 (SEQ ID NO.:5) was chosen as a representative of the PSP94 family based on previous encouraging results of tumor growth inhibition observed in animals.

Test compound. The wild type amino acid sequence of PCK3145 has been disclosed, for example, in international application No.: PCT/CA01/01463 and is defined herein in SEQ ID NO.: 5. A PCK3145 derivative has been generated by attaching an acetylaminomethyl group to the sulfur atom of each of the three cysteines of PCK3145. These groups stabilize the compound by preventing formation of peptide dimers or polymer by blocking the sulfhydryl group of cysteines. This PCK3145 derivative is defined in SEQ ID NO.: 7. The drug was manufactured by Multiple Peptide Systems (3550) (General Atomics Court, San Diego, Calif.) using standard solid-phase peptide chemistry and lyophilized into a powder. Other type of synthesis or manufacture method may however be performed to make a peptide or polypeptide of the invention. Other PCK3145 derivatives, analogs and fragments (e.g., SEQ IDs NO: 88, 98, etc.) may be generated similarly.

The reconstituted drug used in the present example is made from a solution containing a 20 mg/mL of PCK3145 derivative (SEQ ID NO.:5 derivative); SEQ ID NO.: 7, in a phosphate buffer at pH 7.4 for dilution in sterile saline (0.9% NaCl, BP) prior to intravenous administration. The solutions is filled into Type 1 glass vials, stoppered with Teflon®-faced butyl stoppers, and sealed with flip-off seals.

The drug was administered to patients characterized as having metastatic adenocarcinoma of the prostate, stage 1V prostatic cancer and as having a metastatic hormone resistant prostatic cancer.

Biological samples were drawn during different time points. Plasma samples were placed on dry ice and stored frozen (approximately −70° C.) and subsequently analyzed for total MM P-9 levels.

MMP-9 assay methodology. An Elisa assay measuring total MMP-9, i.e., human active and pro-MMP-9, (Quantikine®, Cat. No.: DMP900, R&D Systems Inc.) was performed on plasma-heparin samples. Plasma samples have been collected from individuals at day 1 (before treatment) and at day 27 of each treatment cycle.

The Quantikine® MMP-9 immunoassay is a solid phase ELISA designed to measure total MMP-9 (92 kDa pro- and 82 kDa active forms) in serum, plasma, saliva, urine and cell culture supernatants. It is calibrated with CHO-cells expressed recombinant human pro-MMP-9 and the antibodies were raised against the recombinant factor. Both antibodies also recognize recombinant human active MMP-9. Natural human MMP-9 showed dose-response curves that were parallel to the standard curves obtained using the recombinant Quantikine®) kit standards, indicating that the Quantikine® kit may be used to determine relative mass values of natural human MMP-9.

The assay employs the quantitative sandwich enzyme immunoassay technique. A monoclonal antibody specific for MMP-9 has been pre-coated onto a microplate. Standards and samples are added into the wells, and MMP-9 is thus bound by the immobilized antibody. After washing away unbound substances, an enzyme-linked polyclonal antibody specific for MMP-9 is added to the wells. Following a wash to remove unbound antibody-enzyme reagent, a substrate solution is added to the wells and color develops in proportion to the amount of total MMP-9 (pro and/or active) bound in the initial step. The color development is stopped and the intensity of the color is measured.

Zymography. Zymography is a technique generally used to analyze the activity of matrix metalloproteinases (MMPs) in biological samples. It involves the electrophoretic separation of proteins under denaturing (Sodium Dodecyl Sulfate (SDS)) but non-reducing conditions through a polyacrylamide gel containing gelatin (for example, 10% gel containing 1 mg/ml gelatin for MMP-9 and MMP-2 assays). The resolved proteins are re-natured by exchanging SDS with a non-ionic detergent such as Triton X-100 and the gel is incubated in an incubation buffer for activation of MMP-2 and MMP-9 (for example at 37° C. for 18 hrs). The gel is stained with Coomassie blue and the MMP-2 and MMP-9 bands may be visualized as clear bands against a blue background (i.e., the MMPs degrade the gelatin and are visualized as clear bands; pro MMP-2 is 68 kDa and pro-MMP-9 is 92 kDa). These bands can be quantified using densitometry.

Materials. Cell culture media were obtained from Life Technologies (Burlington, Ontario, Canada) and serum was purchased from Hyclone Laboratories (Logan, Utah). Electrophoresis reagents were purchased from Bio-Rad (Mississauga, Ontario, Canada). The polyclonal (C-1158) and monoclonal (A3) antibodies, used for precipitation and detection, respectively, of VEGFR-2, and the anti-PDGFR pAb (958) were obtained from Santa Cruz Biotechnologies (Santa Cruz, Calif.). Antiphosphotyrosine mAb PY99 was also purchased from Santa Cruz Biotechnologies. Anti-phospho-ERK polyclonal antibodies were from Cell Signaling Technology (Beverly, Mass.). Anti-mouse and anti-rabbit horseradish peroxidase-linked secondary antibodies were purchased from Jackson ImmunoResearch Laboratories (West Grove, Pa.) and enhanced chemiluminescence (ECL) reagents were from Amersham Pharmacia Biotech (Baie d'Urfé, Québec, Canada). Human recombinant PDGF was obtained from R&D Systems (Minneapolis, Minn.). Micro bicinchoninic acid protein assay reagents were from Pierce (Rockford, Ill.). All other reagents were from Sigma-Aldrich Canada.

VEGF production. Vascular endothelial growth factor (isoform 165) was PCRamplified from a pBlast/VEGF plasmid (Invivogen, San Diego, Calif.) and cloned into the pTT vector (14). VEGF was produced following large-scale transient transfection of human 293SFE cells in serum-free medium. The recombinant protein was expressed by the transiently transfected cells and secreted into the medium. The culture was harvested five days after transfection, the medium was clarified by centrifugation at 3,500 g for 10 minutes and filtered through a 0.22 μm membrane. Clarified culture medium was loaded onto a heparin-Sepharose column and the bound VEGF was then eluted using a NaCl gradient in PBS. A buffer exchange for PBS was performed by gel filtration and the final purified material was sterile-filtered, and stored in aliquots at −80° C.

Cell culture. Human umbilical vein endothelial cells (HUVEC) and pulmonary aortic smooth muscle cells (PASMC) were obtained from Clonetics and maintained in endothelial cell basal medium-2, (EBM-2; Clonetics) and smooth muscle medium-2 (SmGM-2; Clonetics), respectively. Cells were cultured at 37° C. under a humidified atmosphere containing 5% CO₂. For experimental purposes, cells were plated in 8 100-mm plastic dishes at 5,000 cells/cm² and were grown to confluence before overnight serum starvation. Cells were treated with vehicle or with a PCK3145 derivative diluted in 0.1 N NaOH, and stimulated with 50 ng/ml VEGF, PDGF or with 1 μM S1P.

Immunoprecipitation and immunoblotting procedures. After treatment, cells were washed once with phosphate-buffered saline (PBS) containing 1 mM sodium orthovanadate and were incubated in the same medium for 1 h at 4° C. The cells were solubilized on ice in lysis buffer (150 mM NaCl, 10 mM Tris-HCl, pH 7.4, 1 mM EDTA, 1 mM EGTA, 0.5% Nonidet P-40, 1% Triton X-100) containing 1 mM sodium orthovanadate. The cells were then scraped from the culture dishes and the resulting lysates were clarified by centrifugation at 10,000 g for 10 min. Protein concentrations were determined using the micro bicinchoninic acid method. For immunoprecipitation studies, lysates were clarified by a 1 h incubation at 4° C. with a mixture of Protein A/Protein G Sepharose beads. After removal of the Sepharose beads by low-speed centrifugation, identical amounts of protein (200 μg) from each sample were transferred to fresh tubes and incubated in lysis buffer overnight at 4° C. in the presence of 2 μg/ml of specific antibodies. Immunocomplexes were collected by incubating the mixture with 25 μl (50% suspension) of Protein A—(rabbit primary antibody) or Protein G—(mouse primary antibody) Sepharose beads, for 2 h. Nonspecifically-bound material was removed by washing the beads three times in 1 ml of lysis buffer containing 1 mM sodium orthovanadate, and bound material was solubilized in 25 μl of two-fold concentrated Laemmli sample buffer, boiled 5 min, and resolved by SDS-PAGE. The proteins were transferred onto polyvinylidene difluoride (PVDF) membranes, blocked 1 h at room temperature with Tris-buffered saline/Tween 20 (147 mM NaCl, 20 mM Tris/HCl, pH 7.5, and 0.1% Tween 20) containing 2% bovine serum albumin and incubated overnight at 4° C. with primary antibody. Immunoreactive bands were revealed after a 1 h incubation with horseradish peroxidase-conjugated anti-mouse or anti-rabbit antibodies, and the signals were visualized by enhanced chemiluminescence (Amersham Biosciences, Baie d'Urfée, QC).

Angiogenesis Assays

Rat aortic ring assay: The isolated rat aorta is cut into segments that are placed in culture, in a matrix-containing environment such as Matrigel. Over the next 7-14 days, the explants are monitored for the outgrowth of endothelial (and other) cells as this is affected by the addition of test substances. Quantification is achieved by measurement of the length and abundance of vessel-like extensions from the explant. Use of endothelium-selective reagents such as fluorescein-labeled BSL-1 allows quantification by pixel counts.

Chick aortic arch assay: Aortic arches are dissected from day 12-14 chick embryos and cut into rings similar to those of the rat aorta. When the rings are placed on Matrigel, substantial outgrowth of cells occurs within 48 h, with the formation of vessel-like structures readily apparent. Test substance is added to the medium and quantification of endothelial cell outgrowth is achieved by the use of fluorescein-labeled lectins such as BSL-1 and BSL-B4 or by staining of the cultures with labeled antibodies to CD31. Standard imaging techniques are used for the enumeration of endothelial cells and for delineating the total outgrowth area.

Cornea angiogenesis assay: A pocket is made in the cornea of a rabbit's eye or mice's eye and angiogenesis is stimulated by an angiogenesis inducer (e.g. VEGF) introduced into this pocket. The inducer elicits ingrowth of new vessels from the peripheral limbal vasculature. Slow-release materials such as ELVAX (ethylene vinyl copolymer), Hydron or sponge may be used to introduce test substances into the corneal pocket.

Inhibition of angiogenesis is monitored by the effect of the inhibitor on the locally induced (e.g., sponge implant) angiogenic reaction in the cornea (e.g., VEGF). The test inhibitor may be administered by several administration mode including, orally, systemically, the latter either by bolus injection or, for example, by use of a sustained-release method such as implantation of osmotic pumps loaded with the test inhibitor.

The vascular response is monitored by direct observation throughout the course of the experiment. This may be done by using a slit lamp for the rabbit but needs only a simple stereomicroscope in mice. Visualization of the mouse corneal vasculature may be achieved by injecting India ink or fluorochrome-labeled high-molecular weight dextran. Methods for quantification include measuring the area of vessel penetration, the progress of vessels toward the angiogenic stimulus overtime, or in the case of fluorescence, histogram analysis or pixel counts above a specific (background) threshold.

Cam assay: The CAM of day 7-9 chick embryos is exposed by making a window in the egg shell, and tissue or organ grafts are then placed directly on the CAM. The window is sealed, eggs are reincubated, and the grafts are recovered after an appropriate length of incubation time. The grafts are then scored for growth and vascularization. The angiogenic reaction may be evaluated by ranking the vascularization on a 0 to 4 basis but also using imaging techniques such as the measurement of bifurcation points in a designated area around the test material. Alternatively, an entire egg contents may be used. Test substances are administered by placing them on membranes or on the underside of coverslips and applied to a desired area. Test compounds are assessed by their effect either on the normal development of the CAM vasculature itself or on induced angiogenesis.

Alternatively, fertilized chick embryos are removed from their shell on day 3 or 4, and a methylcellulose disc containing the test compound is implanted on the chorioallantoic membrane. The embryos are examined 48 hours later, if a clear avascular zone appears around the methylcellulose disc, the diameter of that zone is measured. Such avascular zone indicates a compound having an anti-angiogenic activity (U.S. Pat. No. 5,001,116 (col. 7, incorporated herein by reference).

Matrigel endothelial cell tube formation assay: Matrigel (12.5 mg/ml) was thawed at 4° C., and 50 μl were quickly added to each well of a 96-well plate and allowed to solidify for 10 min at 37° C. The wells were then incubated for 18 h at 37° C. with HUVEC (25,000 cells/well). The formation of capillary-like structures was examined microscopically and pictures (50×) were taken using a Retiga 1300 camera and a Zeiss Axiovert S100 microscope. The extent to which capillary-like structures formed in the gel was quantified by analysis of digitized images to determine the thread length of the capillary-like network, using a commercially available image analysis program (Northern Eclipse).

Matrigel plug assay: Matrigel containing test cells or substances is injected subcutaneously, where it solidifies to form a plug. This plug is recovered after 7-21 days in the animal and examined histologically to determine the extent to which blood vessels have entered it. Fluorescence measurement of plasma volume is achieved using fluorescein isothiocyanate (FITC)-labeled dextran 150. Quantification may alternatively be achieved by measuring the amount of hemoglobin contained in the plug.

In another alternative assay (the sponge/Matrigel assay) Matrigel alone is first introduced into the mouse. A sponge or tissue fragment is then inserted into the plug. New vessels are measured by injection of FITC.

Other angiogenesis assays are described, for example, in Staton, C. A. et al., (Int. J. Exp. Path. (2004), 85, 233-248) the entire content of which is incorporated herein by reference.

Biologically active PSP94 family member; Fragments, derivatives and analogues may be prepared by techniques known in the art (recombinant technology, solid phase synthesis, etc.). The biological activity of derivatives, fragments and analogues may be determined by any of the techniques described herein or known in the field to be relevant for any of the biological activity described above.

For example, serum-starved quiescent endothelial cells (HUVEC) may be incubated with different doses of a putative PCK3145 derivative, analog or fragment (e.g., any of SEQ ID NOs.:9 to 98, combinations) for 24 h and then stimulated with VEGF. Cells may be washed with PBS containing NaF/Na₃VO₄ and incubated in the same medium buffer for 1 h at 4° C. The cells may be scraped from the culture dishes and the resulting lysates clarified by centrifugation. SDS-PAGE (sodium dodecyl sulfate polyacrylamide gel electrophoresis) may be performed to separate the proteins. Western blotting and immunodetection may be performed by using anti-phosphoERK and anti-ERK antibodies. The bands may be quantified to determine the level of inhibition of ERK phosphorylation by the putative PCK3145 derivative. An inhibitory effect of VEGF-induced ERK phosphorylation by the putative PCK3145 derivative, analog or fragment means that the derivative is biologically active.

In another example, a matrigel containing a putative PCK3145 derivative, fragment or analog with an angiogenesis-inducer is injected subcutaneously, to an animal. This plug is recovered after 7-21 days from the animal and examined histologically to determine the extent to which blood vessels have entered it. Quantification is performed as described above. A biologically active PCK3145 derivative, fragment or analog is identified by the reduction in the number of blood vessels which have entered the matrigel plug or the extent to which blood vessels have entered it.

A derivative, fragment or analog causing a diminution in the formation or propagation of blood vessel (tubes, capillary-like structures) in an agiogenesis assay described herein is considered to be a biologically active derivative, fragment or analog.

Each putative derivative, fragment or analogue may be tested using this technique or any other techniques described herein or known in the art.

### EXAMPLE 1

### In Vivo MMPs Measurements

MMP-9 Assay Results

Results of MMP-9 levels in patient's plasma, before and after one or more treatment cycle with PCK3145 derivative; SEQ ID NO.: 7 are illustrated in Table 2.

Normal values of healthy volunteers were not determined in this study but lizasa et al., has determined that the normal range of plasma MMP-9 concentrations is about 11.4 to 59.4 ng/ml. Based on theses values, patients were sub-divided into two categories; those having normal value of MMP-9 (below 100 μg/L) and those having an elevated level of MMP-9 (higher than 100 μg/L) at baseline (see column identified as D1C1 in Table2).

In the normal value MMP-9 category (patients identified as E, F, G, H and I), there was no significant decrease in MMP-9 levels after one cycle of treatment (column identified D27C1) compared to baseline levels. For patients E and G, no decrease in MMP-9 levels was observed compared to baseline values even after 2 cycles of treatment (column identified D27C2). There was still no MMP-9 decrease even after 3 cycles of treatment for patient E (D27C3).

In the elevated MMP-9 category (patients identified as A, B, C and D), a significant decrease was observed for each patient after only one cycle of treatment (see column identified as D27C1). For example a decrease of up to 89% in MMP-9 levels was observed for patient A compared to baseline levels. For patient B, the decrease in MMP-9 was 41% after cycle 1. For patients C and D the decrease at cycle 1 was 90% and 34% respectively.

This decrease was maintained for patients B and C who have received more treatment cycles (see columns identified as D27C2, D27C3 and D27C4). For example, at treatment cycle 2, patient B showed a reduction of 64% of its baseline level of MMP-9. A similar reduction was also measured for patient B at treatment cycle 3; i.e., a 65% reduction, and at treatment cycle 4; a 75% reduction. In the case of patient C, a reduction of 76% in MMP-9 levels was measured at cycle 2.

TABLE 2

| | | | | | | |
|---|---|---|---|---|---|---|
| | | | | | | Maximum |
| Patient | D1C1 | D27C1 | D27C2 | D27C3 | D27C4 | Reduction |

Elevated MMP-9: Baseline = 100 μg/L

| | | | | | | |
|---|---|---|---|---|---|---|
| A | 424 | 47.3 | N.A. | N.A. | N.A. | 89% |
| B | 156.5 | 91.6 | 55.6 | 54.5 | 39.4 | 75% |
| C | 155 | 14.9 | 37.7 | N.A. | N.A. | 90% |
| D | 130.2 | 85.2 | N.A. | N.A. | | 34% |

Normal MMP-9: Baseline = 100 μg/L

| | | | | | |
|---|---|---|---|---|---|
| E | 57.5 | 58 | 60.1 | 101.8 | N.A. |
| F | 53.2 | 73.1 | N.A. | N.A. | N.A. |
| G | 33.9 | 45.4 | 189.6 | | N.A. |
| H | 57.0 | 44.0 | 65 | | |
| I | 22.1 | 18.8 | | | |

N.A. = not applicable

### EXAMPLE 2

### Effect on MMP-9 Secretion

In order to support in vivo results described in Example 1, zymography assays and western blots were performed on cell lines incubated with a PCK3145 derivative (SEQ ID NO.:7).

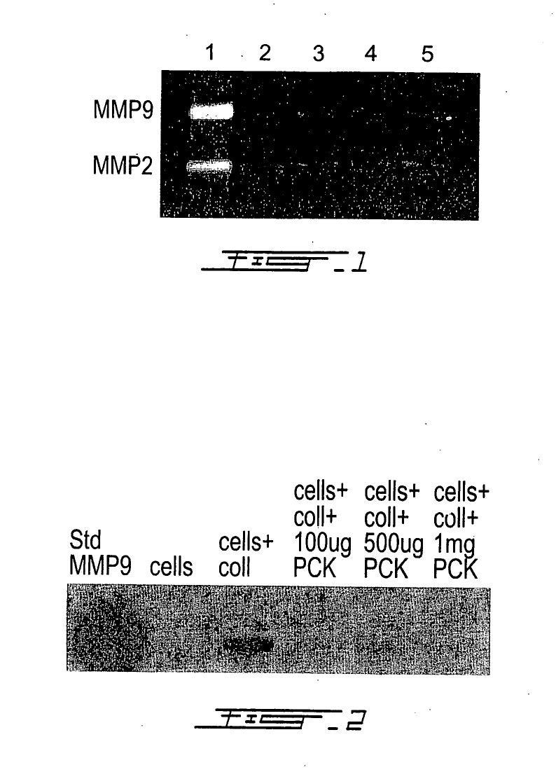

In the experiment presented in FIG. 1, 2.5×10⁵ MatLyLu tumor cells (American Type Culture Collection No.: JHU-5)) were seeded in T-25 flasks containing RPMI with 10% fetal bovine serum (FBS). After overnight incubation, the cells were washed once with serum free medium and treated with various concentrations of the PCK3145 derivative (500 ug/ml and 1 mg/ml) in the presence of 50 ug/ml collagen type-I in serum free RPMI for 72 hrs. Control cells received 50 ug/ml collagen or only serum free medium.

The media were collected after 72 hours of exposure to the PCK3145 derivative and subjected to gelatin zymography. Zymography for MMP-2 and MMP-9 was performed in SDS-polyacrylamide gel electrophoresis (SDS-PAGE) (10%) containing 0.1% gelatin (Invitrogen). Twenty-four microliters of culture media was mixed with non-reducing sample buffer and subjected to electrophoresis without boiling. After electrophoresis, gels were soaked for 30 minutes in 2.5% Triton X-100 solution with 2-3 washing steps. The gels were then incubated for 18 hours at 37° C. in buffer containing 50 mM Tris/HCl, pH 7.6, 50 mM NaCl, 10 mM CaCl₂ and 0.05% Brij-35. After incubation, the gels were stained with 0.2% Coomassie blue and de-stained until clear proteolytic bands appeared. Gels were scanned with Microtek flatbed scanner (Scanmaker 5 software; Microtek lab, Redondo Beach, Calif.). The band intensities were determined using the Image Quant software (version 5.0) from molecular Dynamics.

The MMP-9 and MMP-2 gelatinase zymography standard were purchased from Chemicon (catalogue no. CC073). One nanogram of purified human pro-MMP-2 and pro-MMP-9 standards were used in every gel run.

Results of this experiment are illustrated in FIG. 1 and indicate that PCK3145 derivative treatment of MatLyLu cells resulted in a dose-dependent reduction of MMP-9 secreted in the cell culture media, as detected by zymography.

Western Blot

A separate western blot experiment was performed in which MatLyLu cells were treated with 100 ug/ml, 500 ug/ml and 1 mg/ml of the PCK3145 derivative for 72 hrs. At the end of the experiment, the media were collected and concentrated 5 times using Amicon centrifugal filter devices (3500 molecular weight cut-off).

Twenty five microliters samples were separated on SDS-PAGE gel under reducing conditions using pre-cast gels of 4-12% Bis-Tris (Invitrogen). Following electrophoresis, the proteins were transferred on nitrocellulose membrane. Non-specific binding sites were blocked using 5% skimmed milk in 10 mM phosphate buffer saline (PBS) containing 0.05% Tween-20 for 1 hour at room temperature. The membrane was later incubated with a primary antibody (monoclonal, RDI-MMP-9abm-2A5) at a concentration of 1 ug/ml (in 10 mM PBS, containing 0.5% bovine serum albumin (BSA) and 0.05% Tween-20) for 3 hours at room temperature.

The membranes were washed three times in PBS (5 minutes each wash) to remove non-specific binding and they were incubated with the secondary antibody (Rabbit anti-mouse IgG horseradish peroxidase-conjugated (Dako no. 0260)) at a dilution of 1:5000 for one hour. Detection of specific MMP-9 protein was made by incubating the membrane in ECL™ reagent (electro-chemoluminescence, Roche) and exposing to the X-ray film.

Results of this experiment are illustrated in FIG. 2 and again indicate that treatment of MatLyLu cells PCK3145 derivative resulted in a dose-dependent reduction of MMP-9 levels.

### EXAMPLE 3

### Effect on VEGF Induced MMP-2 Secretion

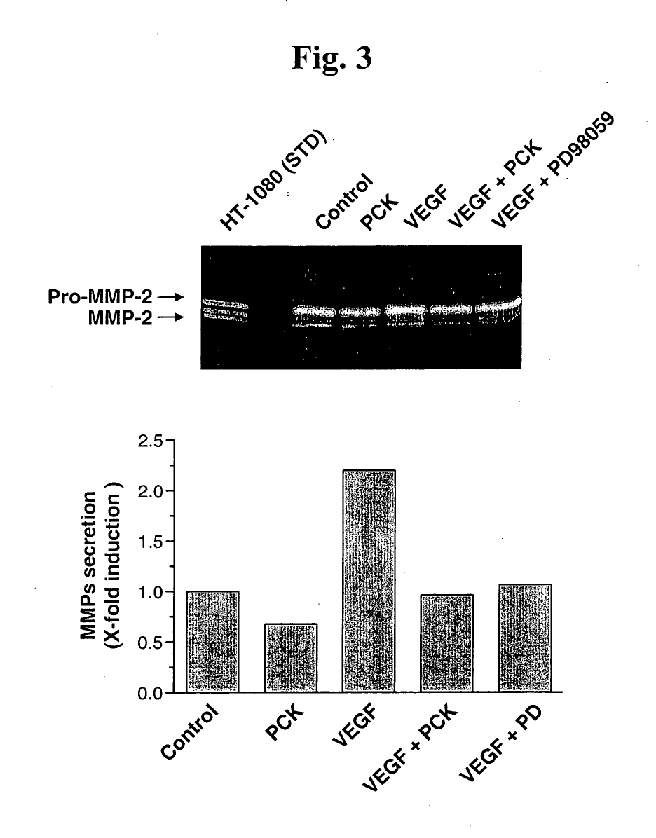

The effect of the PCK3145 derivative (SEQ ID NO.:7) on MMP extracellular levels was assessed by gelatin-zymography in the conditioned media of serum-starved HUVEC. After 16 hours of starvation, HUVEC were stimulated with VEGF in the presence or not of the PCK3145 derivative. A further 24 hours treatment shows that PCK3145 derivative effectively downregulated by approximately 35% the basal proMMP-2 levels in the extracellular media (FIG. 3). Most importantly, the effect of PCK3145 derivative (300 μg/ml) was also observed on VEGF-induced proMMP-2 secretion as the inhibition was of approximately 50%. When these experiments were performed in serum-free media, but in the presence of the MAPK inhibitor PD98059, VEGF-induced proMMP-2 extracellular levels were also significantly decreased. These results suggest that the effect of PCK3145 derivative towards MMP secretion is indeed regulated through a MAPK pathway in endothelial cells.

### EXAMPLE 4

### Phosphorylation of ERK-11/-2 Pathway in Endothelial Cells

VEGF is a strong activator of ERKs (Extracellular-signal-Regulated protein Kinases) 1 and 2 via VEGF receptor 2. In order to test the ability of the PCK3145 derivative in potentially antagonizing VEGF-mediated ERK phosphorylation, serum-starved quiescent endothelial cells (HUVEC) were incubated with vehicle (phosphate-buffered saline (PBS) pH 7.4) or PCK3145 derivative (300 μg/ml) for 24 h and then stimulated with VEGF, bFGF (basic Fibroblast Growth Factor) or S1P (sphingosine-1-phosphate). Cells were washed with PBS containing NaF/Na₃VO₄ and incubated in the same medium buffer for 1 h at 4° C. The cells were scraped from the culture dishes and the resulting lysates clarified by centrifugation. Western blotting and immunodetection using anti-phosphoERK and anti-ERK antibodies was then performed.

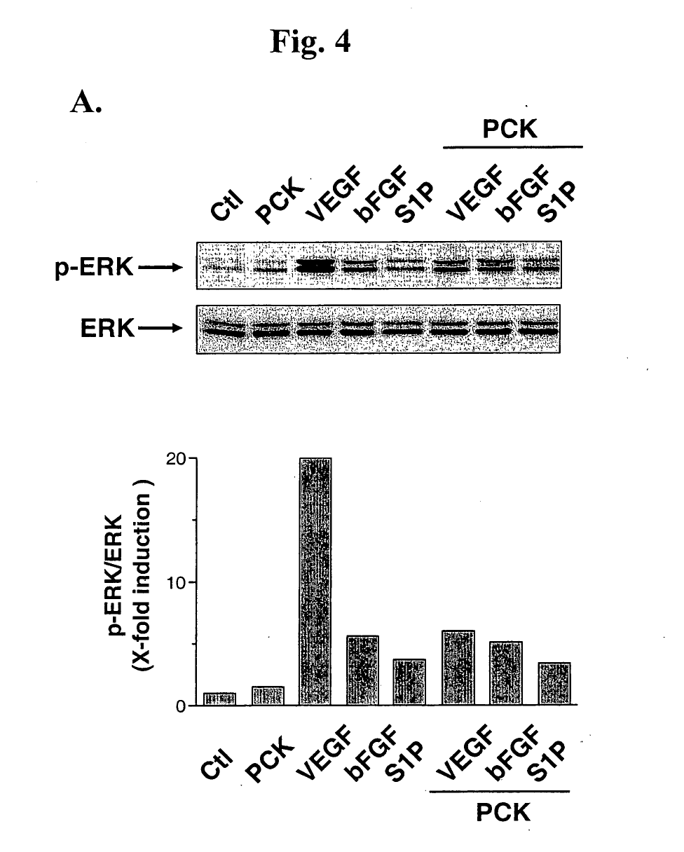

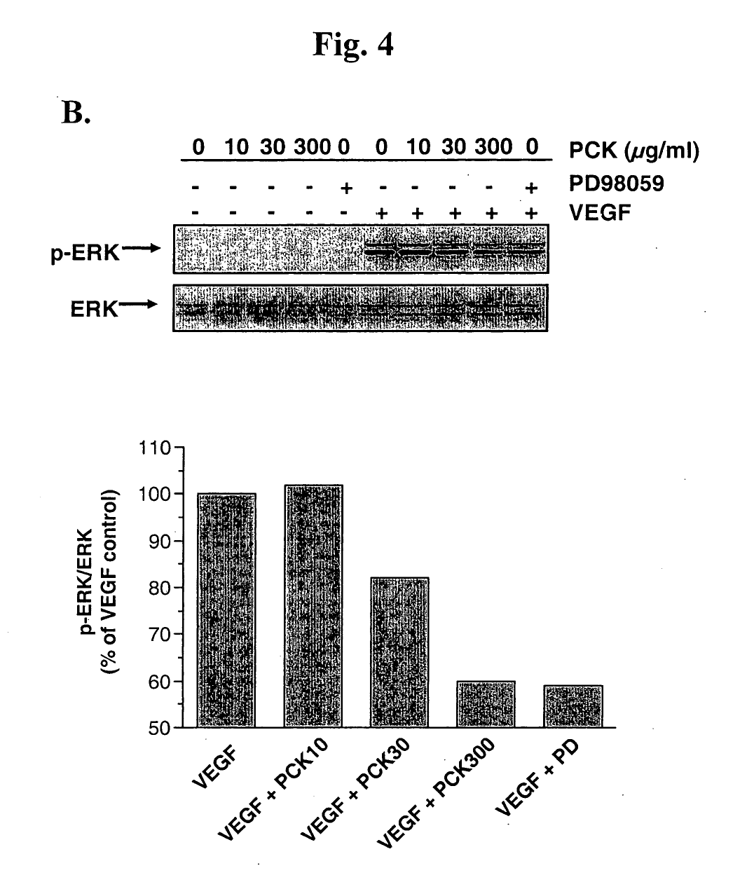

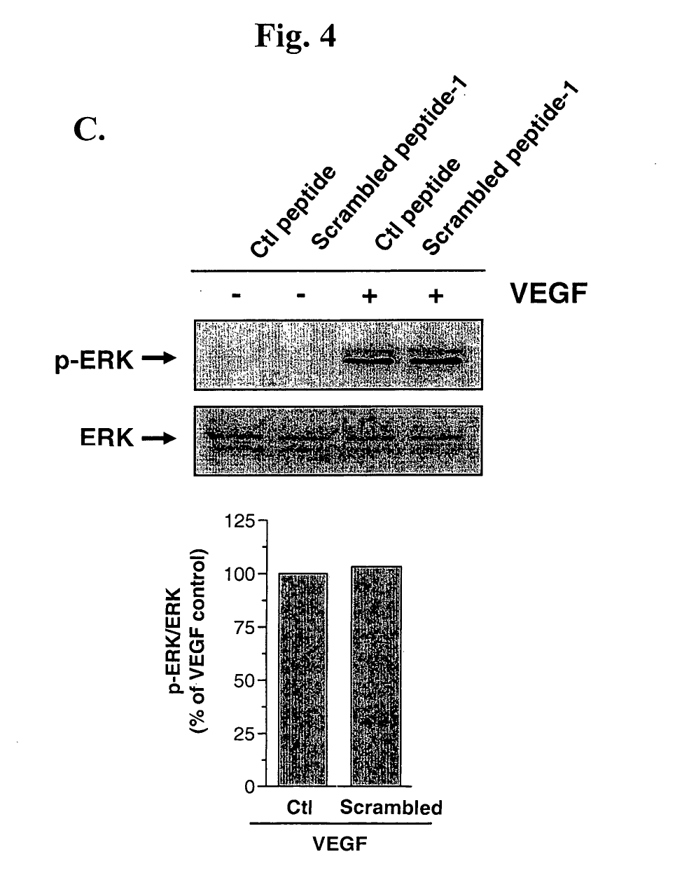

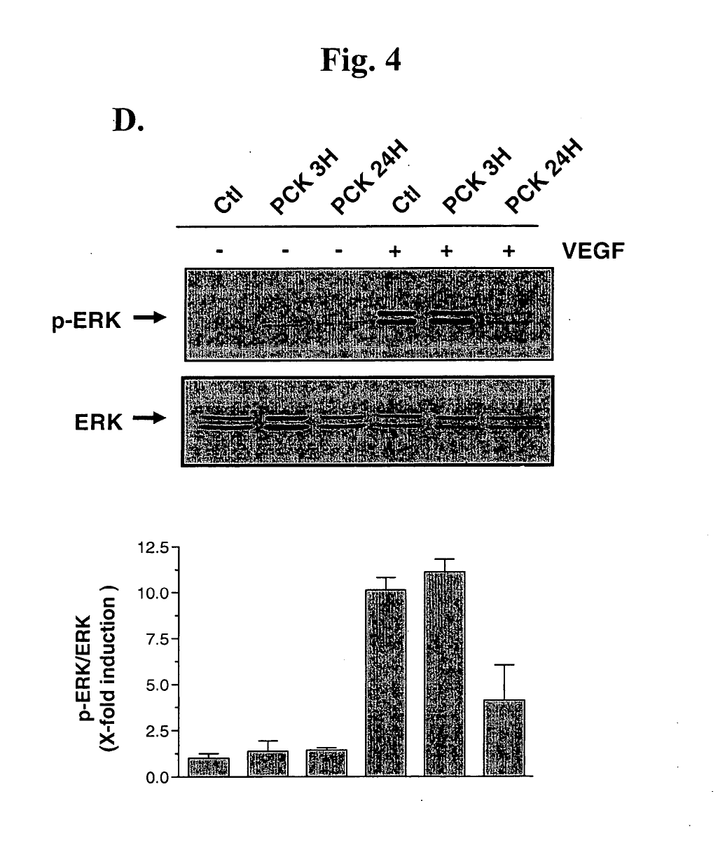

The results show a specific inhibitory effect of the PCK3145 derivative on ERK phosphorylation induced by VEGF (FIG. 4A) but not that induced by bFGF or S1P (FIG. 4A). This inhibitory effect was confirmed for two endothelial cells, HUVEC and BAEC (not shown). The total amount of ERK in each sample of cells was unaffected by the PCK3145 derivative (FIG. 4A). Although, PCK3145 derivative also seemed to stimulate ERK phosphorylation induced by S1P in HUVEC (FIG. 4A), that result was found not statistically significant. Moreover, a dose-response to PCK3145 derivative was found to gradually inhibit the extent of ERK phosphorylation by VEGF (FIG. 4B). The effect of PCK3145 derivative was found comparable to that of PD98059, a documented pharmacological inhibitor of ERK phosphorylation. The lack of effect of a scrambled peptide (SEQ ID No.:99) is demonstrated as a negative control (FIG. 4C). Finally, a time-course of PCK3145 derivative effect is shown at 3 and 24 hrs demonstrating the necessity of a long term action of PCK3145 derivative (FIG. 4D).

### EXAMPLE 5

### Effect on Capillary-Like Structure Formation by HUVEC

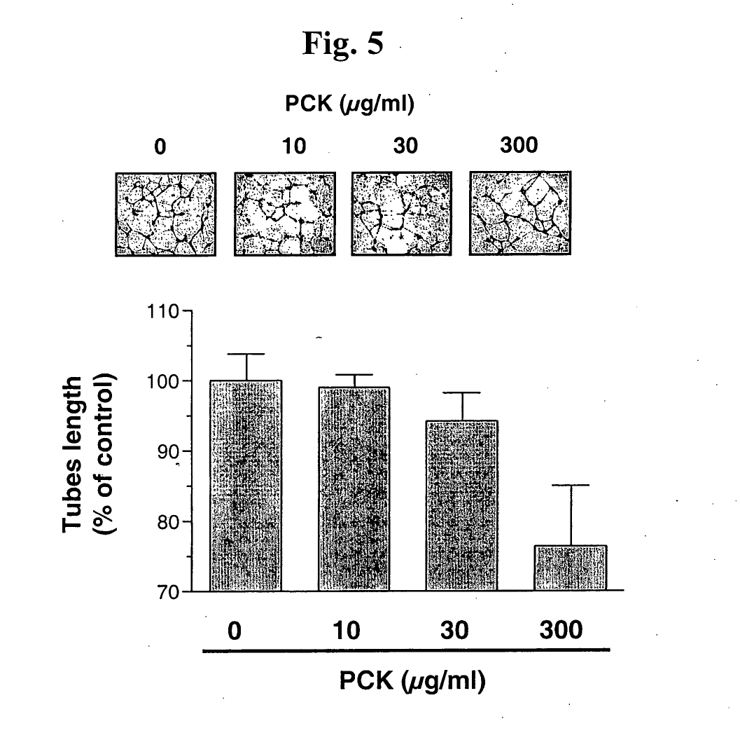

This three dimensional ECM model assay provides physiologically relevant environment for studies of cell morphology, biochemical function, and gene expression in endothelial cells (EC) that can be modulated for instance by tumor growth factors or hypoxic culture conditions. Moreover, proteomic-based approaches to monitor levels of protein expression can also be achieved. When plated on Matrigel, EC have the ability to form capillary-like structures. The extent of capillary-like structures formation (density and size of structures) can be quantified by analysis of digitized images to determine the relative size and area covered by the tube-like network, using an image analysis software (Un-Scan-it, Empix Imaging). HUVEC were trypsinised, counted and seeded on Matrigel. Adhesion to Matrigel was left to proceed for 30 minutes. Treatment with increasing concentrations of the PCK3145 derivative (0-300 μg/ml) was then performed in serum-free media for 24 hours. The extent of capillary-like structure formation was then assessed afterwards. The results show that the PCK3145 derivative negatively affects tubulogenesis (FIG. 5).

### EXAMPLE 6

### Phosphorylation of VEGF Receptors in Endothelial Cells

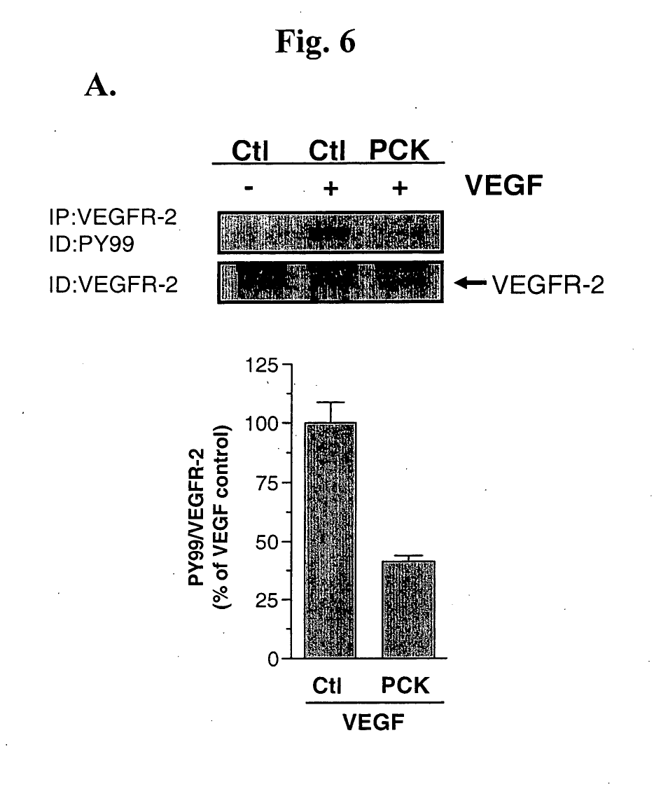

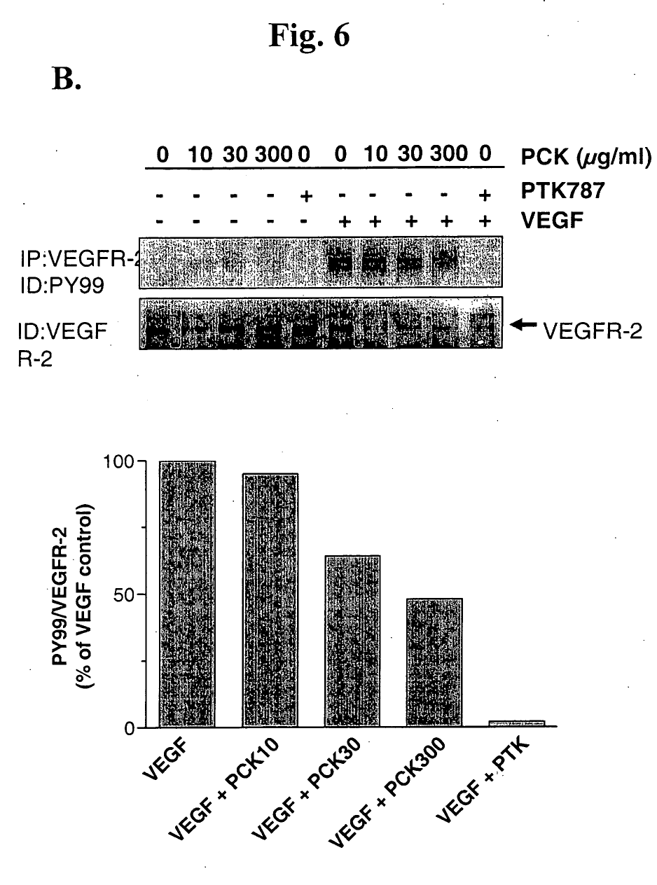

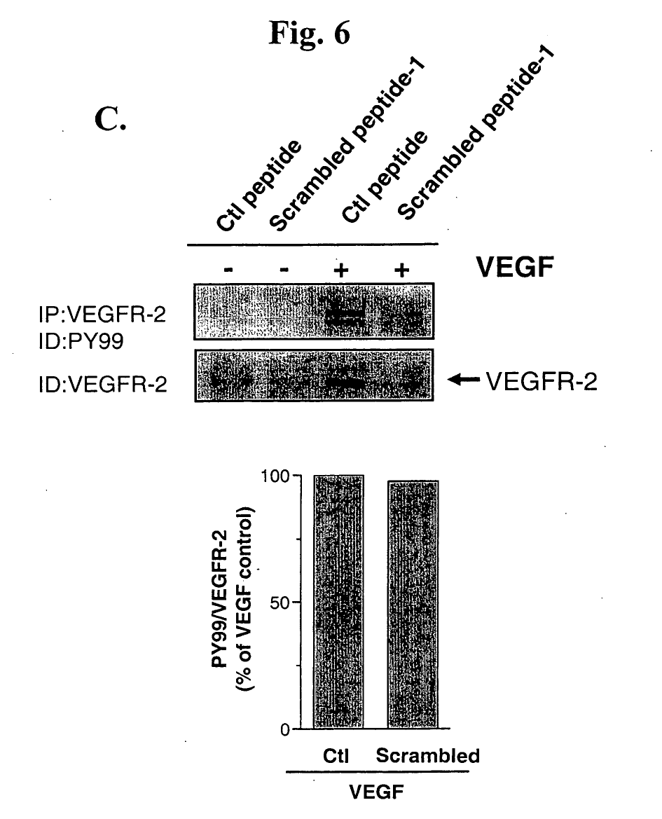

The multifunctionality of VEGF at the cellular level results from its ability to initiate a diverse, complex and integrated network of signaling pathways via its major receptor, VEGFR-2. Thus, the inhibitory effect of the PCK3145 derivative on ERK phosphorylation induced by VEGF was examined to verify whether it was a consequence of an inhibition of the phosphorylation of VEGFR-2. HUVEC were grown, serum-starved, pretreated with the PCK3145 derivative (300 μg/ml; 24 h), and stimulated with VEGF as described in Gingras et al. [Biochem J 348: 273-280, (2000)]. After each treatment, equal amounts of protein were immunoprecipitated with anti-VEGFR-2 polyclonal antibodies and analysed by Western blotting. Results of this experiment show that the PCK3145 derivative inhibited the phosphorylation of VEGFR-2 induced by VEGF in HUVEC (FIG. 6A). This inhibitory effect of the PCK3145 derivative is also shown to be dose-dependent (FIG. 6B), and could be to a certain extent compared to the action of PTK787, a known pharmacological inhibitor of the tyrosine kinase activity associated to the VEGFR-2. Finally, the lack of effect of a scrambled peptide is shown (FIG. 6C) and suggests the specificity of action of the PCK3145 derivative.

### EXAMPLE 7

### Phosphorylation of PDGF Receptors in Smooth Muscle Cells

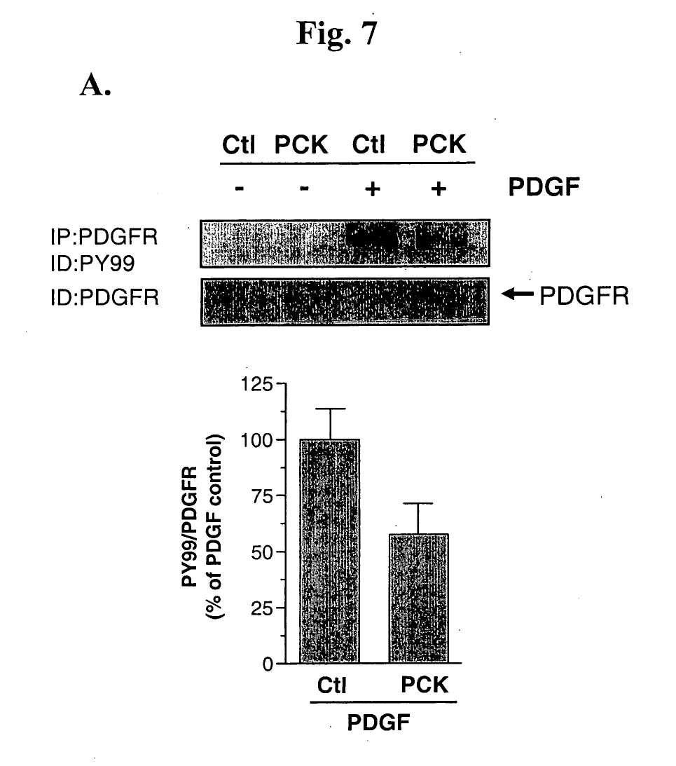

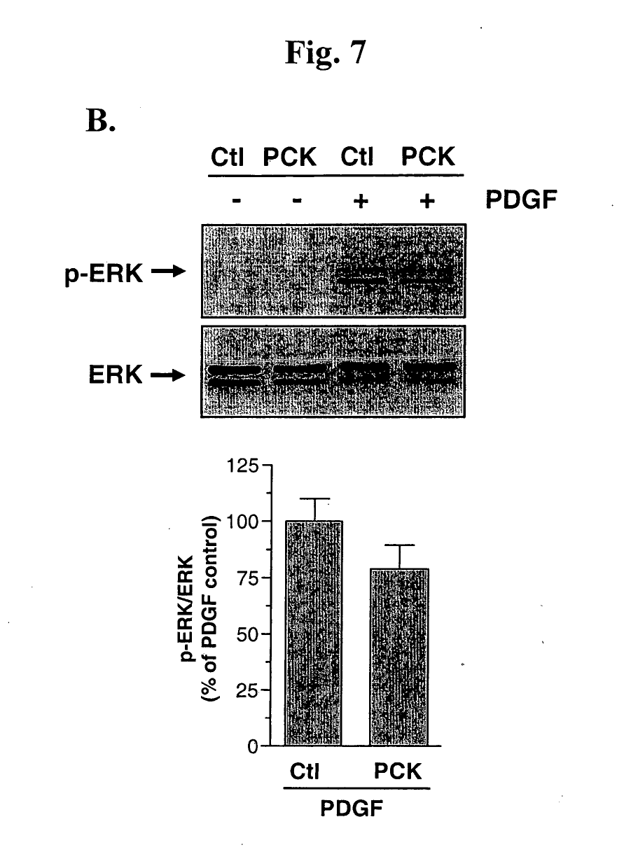

The potential inhibitory action of the PCK3145 derivative towards the tyrosine kinase activity associated to the VEGFR-2 was also tested on the kinase activity associated to another receptor the PDGF receptor (PDGFR) in PASMC (pulmonary aortic smooth muscle cells). Similar treatment of the PCK3145 derivative as for HUVEC was performed. Interestingly, PCK3145 derivative leads to the inhibition of PDGFR phosphorylation induced by PDGF (FIG. 7A), as well as of the PDGF-induced ERK phosphorylation (FIG. 7B).

### EXAMPLE 8

### Intrinsic Effect on ERK Phosphorylation

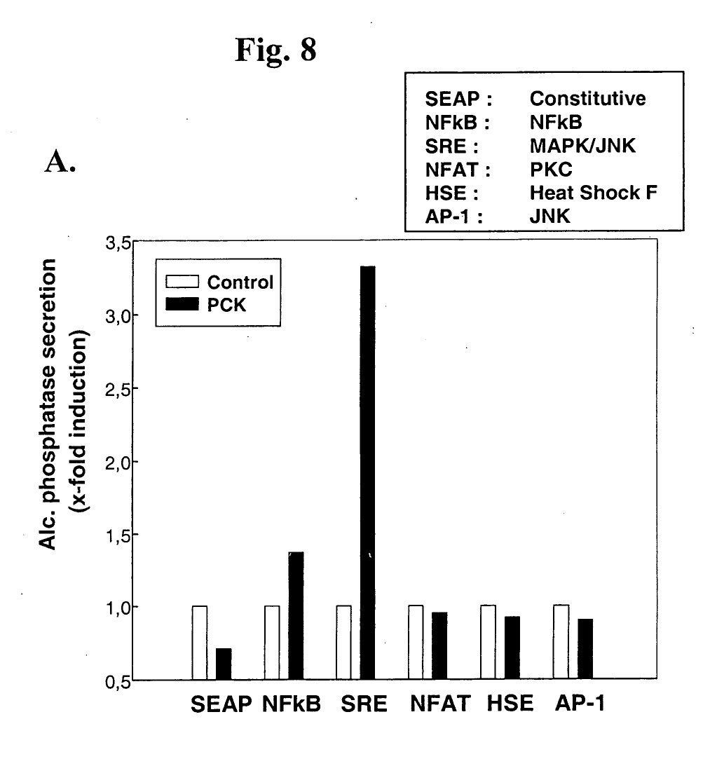

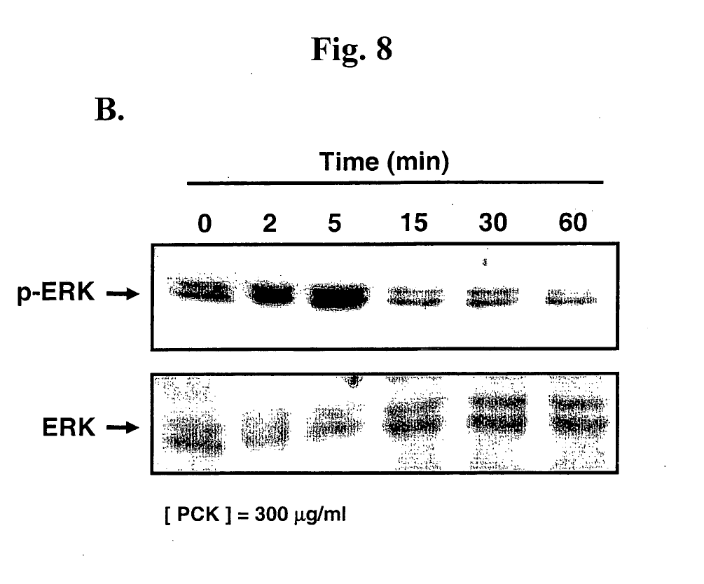

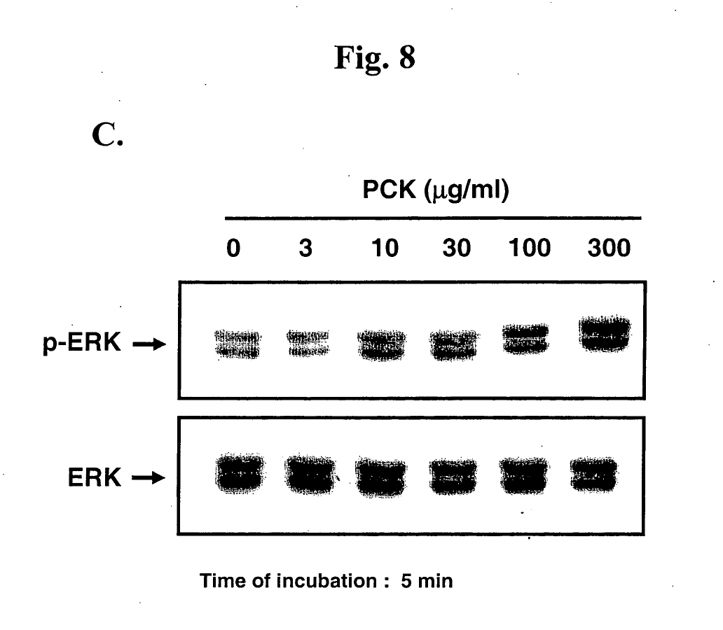

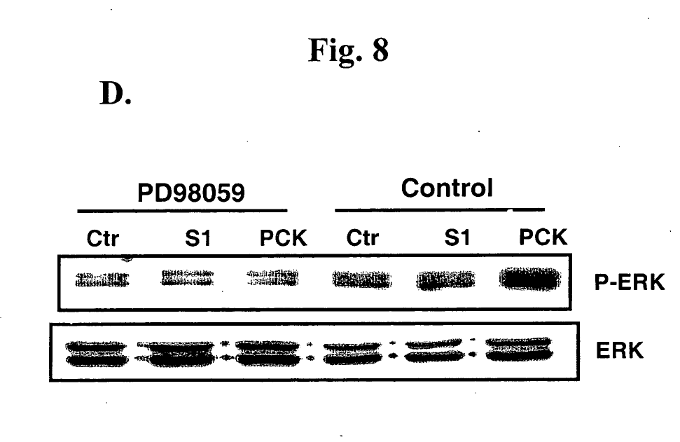

In order to investigate the potential intracellular pathways triggered by the PCK3145 derivative, a gene-reporter assay using the SEAP (Secreted Alkaline Phosphatase) Mercury Profiling Kit (CLONTECH) was performed in glioma cells (U-87). This assay enables the monitoring of transcription factors that are triggered by a particular experimental condition by assaying the alkaline phosphatase activity in the extracellular media. The PCK3145 derivative triggers significantly two pathways: the MAPK/JNK pathway (SRE) and the NFkB pathway (FIG. 8A). The MAPK pathway induction is extremely strong as compared to that of the NFkB pathway. The latter however potentially suggests the involvement of pro-apoptotic pathways that would be triggered by the PCK3145 derivative. Intriguingly, the secretion of the constitutively expressed SEAP was found to be inhibited suggesting a potential effect of the PCK3145 derivative on a more general constitutive secretion pathway. The induction of the MAPK pathway by the PCK3145 derivative is further confirmed by the rapid and transient induction of ERK phosphorylation between 5-10 minutes (FIG. 8B) and is shown to be dose-dependent (FIG. 8C). Finally, the effects of the PCK3145 derivative were also compared to those of a scrambled peptide. These results show that the scrambled peptide was unable to induce ERK phosphorylation comparable to that of the PCK3145 derivative (FIG. 8D). Finally, these results also indicate that the MAPK inhibitor PD98059 antagonized the induction of ERK phosphorylation by the PCK3145 derivative.

### EXAMPLE 9

### Effect on Angiogenesis

Matrigel containing the PCK3145 or its derivative (SEQ ID NO.:5 or SEQ ID NO.:7) is injected subcutaneously to a rat. This solidified plug is recovered after 7-21 days in the animal and examined histologically to determine the extent to which blood vessels have entered the plugs.

In another assay, fertilized chick embryos are removed from their shell on day 3 or 4, and a methylcellulose disc containing PCK3145 (SEQ ID NO.:5 or SEQ ID NO.:7) is implanted on the chorioallantoic membrane. The embryos are examined 48 hours later and the diameter of the avascular zone is measured.

The overall effects of PSP94 family members described herein make them useful for treatment of several diseases in addition to the previously disclosed utility (inhibition of tumor cell growth and skeletal metastasis).

The content of each publication, patent and patent application mentioned in the present application is incorporated herein by reference.

Although the present invention has been described in details herein and illustrated in the accompanying drawings, it is to be understood that the invention is not limited to the embodiments described herein and that various changes and modifications may be effected without departing from the scope or spirit of the present invention.

| SEQUENCE DESCRIPTION: SEQ ID NO: 1: | | | | | | |
|---|---|---|---|---|---|---|
| Ser Cys Tyr Phe Ile Pro Asn Glu Gly Val Pro Gly Asp Ser Thr Arg | | | | | | |
| 1 5 10 15 | | | | | | |
| Lys Cys Met Asp Leu Lys Gly Asn Lys His Pro Ile Asn Ser Glu Trp | | | | | | |
| 20 25 30 | | | | | | |
| Gln Thr Asp Asn Cys Glu Thr Cys Thr Cys Tyr Glu Thr Glu Ile Ser | | | | | | |
| 35 40 45 | | | | | | |
| Cys Cys Thr Leu Val Ser Thr Pro Val Gly Tyr Asp Lys Asp Asn Cys | | | | | | |
| 50 55 60 | | | | | | |
| Gln Arg Ile Phe Lys Lys Glu Asp Cys Lys Tyr Ile Val Val Glu Lys | | | | | | |
| 65 70 75 80 | | | | | | |
| Lys Asp Pro Lys Lys Thr Cys Ser Val Ser Glu Trp Ile Ile | | | | | | |
| 85 90 | | | | | | |
| SEQUENCE DESCRIPTION: SEQ ID NO: 2: | | | | | | |
| Glu Ala Glu Ala Tyr Val Glu Phe Ser Cys Tyr Phe Ile Pro Asn Glu | | | | | | |
| 1 5 10 15 | | | | | | |
| Gly Val Pro Gly Asp Ser Thr Arg Lys Cys Met Asp Leu Lys Gly Asn | | | | | | |
| 20 25 30 | | | | | | |
| Lys His Pro Ile Asn Ser Glu Trp Gln Thr Asp Asn Cys Glu Thr Cys | | | | | | |
| 35 40 45 | | | | | | |
| Thr Cys Tyr Glu Thr Glu Ile Ser Cys Cys Thr Leu Val Ser Thr Pro | | | | | | |
| 50 55 60 | | | | | | |
| Val Gly Tyr Asp Lys Asp Asn Cys Gln Arg Ile Phe Lys Lys Glu Asp | | | | | | |
| 65 70 75 80 | | | | | | |
| Cys Lys Tyr Ile Val Val Glu Lys Lys Asp Pro Lys Lys Thr Cys Ser | | | | | | |
| 85 90 95 | | | | | | |
| Val Ser Glu Trp Ile Ile | | | | | | |
| 100 | | | | | | |
| SEQUENCE DESCRIPTION: SEQ ID NO: 3: | | | | | | |
| Tyr Thr Cys Ser Val Ser Glu Pro Gly Ile | | | | | | |
| 1 5 10 | | | | | | |
| SEQUENCE DESCRIPTION: SEQ ID NO: 4: | | | | | | |
| Asn Glu Gly Val Pro Gly Asp Ser Thr Arg Lys Cys Met Asp Leu | | | | | | |
| 1 5 10 15 | | | | | | |
| SEQUENCE DESCRIPTION: SEQ ID NO: 5: | | | | | | |
| Glu Trp Gln Thr Asp Asn Cys Glu Thr Cys Thr Cys Tyr Glu Thr | | | | | | |
| 1 5 10 15 | | | | | | |
| SEQUENCE DESCRIPTION: SEQ ID NO: 6: | | | | | | |
| Ile Val Val Glu Lys Lys Asp Pro Lys Lys Thr Cys Ser Val Ser Glu | | | | | | |
| 1 5 10 15 | | | | | | |
| Trp Ile Ile | | | | | | |
| SEQUENCE DESCRIPTION: SEQ ID NO: 7 (an acetylaminomethyl group may be | | | | | | |
| attached to the sulfur atom of cysteine 7, of cysteine 10 and/or of | | | | | | |
| cysteine 12) | | | | | | |
| Glu Trp Gln Thr Asp Asn Cys Glu Thr Cys Thr Cys Tyr Glu Thr | | | | | | |
| 1 5 10 15 | | | | | | |
| SEQUENCE DESCRIPTION: SEQ ID NO: 8: | | | | | | |
| TCATGCTATT | TCATACCTAA | TGAGGGAGTT | CCAGGAGATT | CAACCAGGAA | ATGCATGGAT | 60 |
| CTCAAAGGAA | ACAAACACCC | AATAAACTCG | GAGTGGCAGA | CTGACAACTG | TGAGACATGC | 120 |
| ACTTGCTACG | AAACAGAAAT | TTCATGTTGC | ACCCTTGTTT | CTACACCTGT | GGGTTATGAC | 180 |
| AAAGACAACT | GCCAAAGAAT | CTTCAAGAAG | GAGGACTGCA | AGTATATCGT | GGTGGAGAAG | 240 |
| AAGGACCCAA | AAAAGACCTG | TTCTGTCAGT | GAATGGATAA | TCTAA | | 285 |

| SEQUENCE DESCRIPTION: SEQ ID NO: 9: | | |
|---|---|---|
| Glu Trp Gln Thr Asp Asn Cys Glu Thr Cys Thr Cys Tyr Glu Thr Glu | | |
| 1 5 10 15 | | |
| SEQUENCE DESCRIPTION: SEQ ID NO: 10: | | |
| Glu Trp Gln Thr Asp Asn Cys Glu Thr Cys Thr Cys Tyr Glu Thr Glu | | |
| 1 5 10 15 | | |
| Ile | | |
| SEQUENCE DESCRIPTION: SEQ ID NO: 11: | | |
| Glu Trp Gln Thr Asp Asn Cys Glu Thr Cys Thr Cys Tyr Glu Thr Glu | | |
| 1 5 10 15 | | |
| Ile Ser | | |
| SEQUENCE DESCRIPTION: SEQ ID NO: 12: | | |
| Glu Trp Gln Thr Asp Asn Cys Glu Thr Cys Thr Cys Tyr Glu Thr Glu | | |
| 1 5 10 15 | | |
| Ile Ser Cys | | |
| SEQUENCE DESCRIPTION: SEQ ID NO: 13: | | |
| Glu Trp Gln Thr Asp Asn Cys Glu Thr Cys Thr Cys Tyr Glu Thr Glu | | |
| 1 5 10 15 | | |
| Ile Ser Cys Cys | | |
| 20 | | |
| SEQUENCE DESCRIPTION: SEQ ID NO: 14: | | |
| Glu Trp Gln Thr Asp Asn Cys Glu Thr Cys Thr Cys Tyr Glu Thr Glu | | |
| 1 5 10 15 | | |
| Ile Ser Cys Cys Thr | | |
| 20 | | |
| SEQUENCE DESCRIPTION: SEQ ID NO: 15: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu | | |
| 20 | | |
| SEQUENCE DESCRIPTION: SEQ ID NO: 16: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val | | |
| 20 | | |
| SEQUENCE DESCRIPTION: SEQ ID NO: 17: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser | | |
| 20 | | |
| SEQUENCE DESCRIPTION: SEQ ID NO: 18: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr | | |
| 20 25 | | |
| SEQUENCE DESCRIPTION: SEQ ID NO: 19: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro | | |
| 20 25 | | |
| SEQUENCE DESCRIPTION: SEQ ID NO: 20: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val | | |
| 20 25 | | |
| SEQUENCE DESCRIPTION: SEQ ID NO: 21: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly | | |
| 20 25 | | |
| SEQUENCE DESCRIPTION: SEQ ID NO: 22: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr | | |
| 20 25 | | |
| SEQUENCE DESCRIPTION: SEQ ID NO: 23: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp | | |
| 20 25 30 | | |
| SEQUENCE DESCRIPTION: SEQ ID NO: 24: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys | | |
| 20 25 30 | | |
| SEQUENCE DESCRIPTION: SEQ ID NO: 25: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| SEQUENCE DESCRIPTION: SEQ ID NO: 26: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn | | |
| SEQUENCE DESCRIPTION: SEQ ID NO: 27: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys | | |
| SEQUENCE DESCRIPTION: SEQ ID NO: 28: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln | | |
| 35 | | |
| SEQUENCE DESCRIPTION: SEQ ID NO: 29: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg | | |
| 35 | | |
| SEQUENCE DESCRIPTION: SEQ ID NO: 30: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile | | |
| 35 | | |
| SEQUENCE DESCRIPTION: SEQ ID NO: 31: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe | | |
| 35 | | |
| SEQUENCE DESCRIPTION: SEQ ID NO: 32: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys | | |
| 35 | | |
| SEQUENCE DESCRIPTION: SEQ ID NO: 33: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys | | |
| 35 40 | | |
| SEQUENCE DESCRIPTION: SEQ ID NO: 34: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu | | |
| 35 40 | | |
| SEQUENCE DESCRIPTION: SEQ ID NO: 35: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp | | |
| 35 40 | | |
| SEQUENCE DESCRIPTION: SEQ ID NO: 36: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys | | |
| 35 40 | | |
| SEQUENCE DESCRIPTION: SEQ ID NO: 37: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys | | |
| 35 40 | | |
| SEQUENCE DESCRIPTION: SEQ ID NO: 38: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr | | |
| 35 40 45 | | |
| SEQUENCE DESCRIPTION: SEQ ID NO: 39: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile | | |
| 35 40 45 | | |
| SEQUENCE DESCRIPTION: SEQ ID NO: 40: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val | | |
| 35 40 45 | | |
| SEQUENCE DESCRIPTION: SEQ ID NO: 41: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| SEQUENCE DESCRIPTION: SEQ ID NO: 42: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu | | |
| SEQUENCE DESCRIPTION: SEQ ID NO: 43: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys | | |
| 50 | | |
| SEQUENCE DESCRIPTION: SEQ ID NO: 44: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys | | |
| 50 | | |
| SEQUENCE DESCRIPTION: SEQ ID NO: 45: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp | | |
| 50 | | |
| SEQUENCE DESCRIPTION: SEQ ID NO: 46: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro | | |
| 50 | | |
| SEQUENCE DESCRIPTION: SEQ ID NO: 47: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys | | |
| 50 | | |
| SEQUENCE DESCRIPTION: SEQ ID NO: 48: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys | | |
| 50 55 | | |
| SEQUENCE DESCRIPTION: SEQ ID NO: 49: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr | | |
| 50 55 | | |
| SEQUENCE DESCRIPTION: SEQ ID NO: 50: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys | | |
| 50 55 | | |
| SEQUENCE DESCRIPTION: SEQ ID NO: 51: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser | | |
| 50 55 | | |
| SEQUENCE DESCRIPTION: SEQ ID NO: 52: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val | | |
| 50 55 | | |
| SEQUENCE DESCRIPTION: SEQ ID NO: 53: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val Ser | | |
| 50 55 60 | | |
| SEQUENCE DESCRIPTION: SEQ ID NO: 54: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val Ser Glu | | |
| 50 55 60 | | |
| SEQUENCE DESCRIPTION: SEQ ID NO: 55: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val Ser Glu Trp | | |
| 50 55 60 | | |
| SEQUENCE DESCRIPTION: SEQ ID NO: 56: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val Ser Glu Trp Ile | | |
| 50 55 60 | | |
| SEQUENCE DESCRIPTION: SEQ ID NO: 57: | | |
| Glu Trp Gln Thr Asp Asn Cys Glu Thr Cys Thr Cys Tyr Glu Thr Glu | | |
| 1 5 10 15 | | |
| Ile Ser Cys Cys Thr Leu Val Ser Thr Pro Val Gly Tyr Asp Lys Asp | | |
| 20 25 30 | | |
| Asn Cys Gln Arg Ile Phe Lys Lys Glu Asp Cys Lys Tyr Ile Val Val | | |
| 35 40 45 | | |
| Glu Lys Lys Asp Pro Lys Lys Thr Cys Ser Val Ser Glu Trp Ile Ile | | |
| 50 55 60 | | |
| SEQUENCE DESCRIPTION: SEQ ID NO: 58: | | |
| Ser Glu Trp Gln Thr Asp Asn Cys Glu Thr Cys Thr Cys Tyr Glu Thr | | |
| 1 5 10 15 | | |
| SEQUENCE DESCRIPTION: SEQ ID NO: 59: | | |
| Asn Ser Glu Trp Gln Thr Asp Asn Cys Glu Thr Cys Thr Cys Tyr Glu | | |
| 1 5 10 15 | | |
| Thr | | |
| SEQUENCE DESCRIPTION: SEQ ID NO: 60: | | |
| Ile Asn Ser Glu Trp Gln Thr Asp Asn Cys Glu Thr Cys Thr Cys Tyr | | |
| 1 5 10 15 | | |
| Glu Thr | | |
| SEQUENCE DESCRIPTION: SEQ ID NO: 61: | | |
| Pro Ile Asn Ser Glu Trp Gln Thr Asp Asn Cys Glu Thr Cys Thr Cys | | |
| 1 5 10 15 | | |
| Tyr Glu Thr | | |
| SEQUENCE DESCRIPTION: SEQ ID NO: 62: | | |
| His Pro Ile Asn Ser Glu Trp Gln Thr Asp Asn Cys Glu Thr Cys Thr | | |
| 1 5 10 15 | | |
| Cys Tyr Glu Thr | | |
| 20 | | |
| SEQUENCE DESCRIPTION: SEQ ID NO: 63: | | |
| Lys His Pro Ile Asn Ser Glu Trp Gln Thr Asp Asn Cys Glu Thr Cys | | |
| 1 5 10 15 | | |
| Thr Cys Tyr Glu Thr | | |
| 20 | | |
| SEQUENCE DESCRIPTION: SEQ ID NO: 64: | | |
| Asn Lys His Pro Ile Asn Ser Glu Trp Gln Thr Asp Asn Cys Glu Thr | | |
| 1 5 10 15 | | |
| Cys Thr Cys Tyr Glu Thr | | |
| 20 | | |
| SEQUENCE DESCRIPTION: SEQ ID NO: 65: | | |
| Gly Asn Lys His Pro Ile Asn Ser Glu Trp Gln Thr Asp Asn Cys Glu | | |
| 1 5 10 15 | | |
| Thr Cys Thr Cys Tyr Glu Thr | | |
| 20 | | |
| SEQUENCE DESCRIPTION: SEQ ID NO: 66: | | |
| Lys Gly Asn Lys His Pro Ile Asn Ser Glu Trp Gln Thr Asp Asn Cys | | |
| 1 5 10 15 | | |
| Glu Thr Cys Thr Cys Tyr Glu Thr | | |
| 20 | | |
| SEQUENCE DESCRIPTION: SEQ ID NO: 67: | | |
| Leu Lys Gly Asn Lys His Pro Ile Asn Ser Glu Trp Gln Thr Asp Asn | | |
| 1 5 10 15 | | |
| Cys Glu Thr Cys Thr Cys Tyr Glu Thr | | |
| 20 25 | | |
| SEQUENCE DESCRIPTION: SEQ ID NO: 68: | | |
| Asp Leu Lys Gly Asn Lys His Pro Ile Asn Ser Glu Trp Gln Thr Asp | | |
| 1 5 10 15 | | |
| Asn Cys Glu Thr Cys Thr Cys Tyr Glu Thr | | |
| 20 25 | | |
| SEQUENCE DESCRIPTION: SEQ ID NO: 69: | | |
| Met Asp Leu Lys Gly Asn Lys His Pro Ile Asn Ser Glu Trp Gln Thr | | |
| 1 5 10 15 | | |
| Asp Asn Cys Glu Thr Cys Thr Cys Tyr Glu Thr | | |
| 20 25 | | |
| SEQUENCE DESCRIPTION: SEQ ID NO: 70: | | |
| Cys Met Asp Leu Lys Gly Asn Lys His Pro Ile Asn Ser Glu Trp Gln | | |
| 1 5 10 15 | | |
| Thr Asp Asn Cys Glu Thr Cys Thr Cys Tyr Glu Thr | | |
| 20 25 | | |
| SEQUENCE DESCRIPTION: SEQ ID NO: 71: | | |
| Lys Cys Met Asp Leu Lys Gly Asn Lys His Pro Ile Asn Ser Glu Trp | | |
| 1 5 10 15 | | |
| Gln Thr Asp Asn Cys Glu Thr Cys Thr Cys Tyr Glu Thr | | |
| 20 25 | | |
| SEQUENCE DESCRIPTION: SEQ ID NO: 72: | | |
| Arg Lys Cys Met Asp Leu Lys Gly Asn Lys His Pro Ile Asn Ser Glu | | |
| 1 5 10 15 | | |
| Trp Gln Thr Asp Asn Cys Glu Thr Cys Thr Cys Tyr Glu Thr | | |
| 20 25 30 | | |
| SEQUENCE DESCRIPTION: SEQ ID NO: 73: | | |
| Thr Arg Lys Cys Met Asp Leu Lys Gly Asn Lys His Pro Ile Asn Ser | | |
| 1 5 10 15 | | |
| Glu Trp Gln Thr Asp Asn Cys Glu Thr Cys Thr Cys Tyr Glu Thr | | |
| 20 25 30 | | |
| SEQUENCE DESCRIPTION: SEQ ID NO: 74: | | |
| Ser Thr Arg Lys Cys Met Asp Leu Lys Gly Asn Lys His Pro Ile Asn | | |
| 1 5 10 15 | | |
| Ser Glu Trp Gln Thr Asp Asn Cys Glu Thr Cys Thr Cys Tyr Glu Thr | | |
| 20 25 30 | | |
| SEQUENCE DESCRIPTION: SEQ ID NO: 75: | | |
| Asp Ser Thr Arg Lys Cys Met Asp Leu Lys Gly Asn Lys His Pro Ile | | |
| 1 5 10 15 | | |
| Asn Ser Glu Trp Gln Thr Asp Asn Cys Glu Thr Cys Thr Cys Tyr Glu | | |
| 20 25 30 | | |
| Thr | | |
| SEQUENCE DESCRIPTION: SEQ ID NO: 76: | | |
| Gly Asp Ser Thr Arg Lys Cys Met Asp Leu Lys Gly Asn Lys His Pro | | |
| 1 5 10 15 | | |
| Ile Asn Ser Glu Trp Gln Thr Asp Asn Cys Glu Thr Cys Thr Cys Tyr | | |
| 20 25 30 | | |
| Glu Thr | | |
| SEQUENCE DESCRIPTION: SEQ ID NO: 77: | | |
| Pro Gly Asp Ser Thr Arg Lys Cys Met Asp Leu Lys Gly Asn Lys His | | |
| 1 5 10 15 | | |
| Pro Ile Asn Ser Glu Trp Gln Thr Asp Asn Cys Glu Thr Cys Thr Cys | | |
| 20 25 30 | | |
| Tyr Glu Thr | | |
| 35 | | |
| SEQUENCE DESCRIPTION: SEQ ID NO: 78: | | |
| Val Pro Gly Asp Ser Thr Arg Lys Cys Met Asp Leu Lys Gly Asn Lys | | |
| 1 5 10 15 | | |
| His Pro Ile Asn Ser Glu Trp Gln Thr Asp Asn Cys Glu Thr Cys Thr | | |
| 20 25 30 | | |
| Cys Tyr Glu Thr | | |
| 35 | | |
| SEQUENCE DESCRIPTION: SEQ ID NO: 79: | | |
| Gly Val Pro Gly Asp Ser Thr Arg Lys Cys Met Asp Leu Lys Gly Asn | | |
| 1 5 10 15 | | |
| Lys His Pro Ile Asn Ser Glu Trp Gln Thr Asp Asn Cys Glu Thr Cys | | |
| 20 25 30 | | |
| Thr Cys Tyr Glu Thr | | |
| 35 | | |
| SEQUENCE DESCRIPTION: SEQ ID NO: 80: | | |
| Glu Gly Val Pro Gly Asp Ser Thr Arg Lys Cys Met Asp Leu Lys Gly | | |
| 1 5 10 15 | | |
| Asn Lys His Pro Ile Asn Ser Glu Trp Gln Thr Asp Asn Cys Glu Thr | | |
| 20 25 30 | | |
| Cys Thr Cys Tyr Glu Thr | | |
| 35 | | |
| SEQUENCE DESCRIPTION: SEQ ID NO: 81: | | |
| Asn Glu Gly Val Pro Gly Asp Ser Thr Arg Lys Cys Met Asp Leu Lys | | |
| 1 5 10 15 | | |
| Gly Asn Lys His Pro Ile Asn Ser Glu Trp Gln Thr Asp Asn Cys Glu | | |
| 20 25 30 | | |
| Thr Cys Thr Cys Tyr Glu Thr | | |
| 35 | | |
| SEQUENCE DESCRIPTION: SEQ ID NO: 82: | | |
| Pro Asn Glu Gly Val Pro Gly Asp Ser Thr Arg Lys Cys Met Asp Leu | | |
| 1 5 10 15 | | |
| Lys Gly Asn Lys His Pro Ile Asn Ser Glu Trp Gln Thr Asp Asn Cys | | |
| 20 25 30 | | |
| Glu Thr Cys Thr Cys Tyr Glu Thr | | |
| 35 40 | | |
| SEQUENCE DESCRIPTION: SEQ ID NO: 83: | | |
| Ile Pro Asn Glu Gly Val Pro Gly Asp Ser Thr Arg Lys Cys Met Asp | | |
| 1 5 10 15 | | |
| Leu Lys Gly Asn Lys His Pro Ile Asn Ser Glu Trp Gln Thr Asp Asn | | |
| 20 25 30 | | |
| Cys Glu Thr Cys Thr Cys Tyr Glu Thr | | |
| 35 40 | | |
| SEQUENCE DESCRIPTION: SEQ ID NO: 84: | | |
| Phe Ile Pro Asn Glu Gly Val Pro Gly Asp Ser Thr Arg Lys Cys Met | | |
| 1 5 10 15 | | |
| Asp Leu Lys Gly Asn Lys His Pro Ile Asn Ser Glu Trp Gln Thr Asp | | |
| 20 25 30 | | |
| Asn Cys Glu Thr Cys Thr Cys Tyr Glu Thr | | |
| 35 40 | | |
| SEQUENCE DESCRIPTION: SEQ ID NO: 85: | | |
| Tyr Phe Ile Pro Asn Glu Gly Val Pro Gly Asp Ser Thr Arg Lys Cys | | |
| 1 5 10 15 | | |
| Met Asp Leu Lys Gly Asn Lys His Pro Ile Asn Ser Glu Trp Gln Thr | | |
| 20 25 30 | | |
| Asp Asn Cys Glu Thr Cys Thr Cys Tyr Glu Thr | | |
| 35 40 | | |
| SEQUENCE DESCRIPTION: SEQ ID NO: 86: | | |
| Cys Tyr Phe Ile Pro Asn Glu Gly Val Pro Gly Asp Ser Thr Arg Lys | | |
| 1 5 10 15 | | |
| Cys Met Asp Leu Lys Gly Asn Lys His Pro Ile Asn Ser Glu Trp Gln | | |
| 20 25 30 | | |
| Thr Asp Asn Cys Glu Thr Cys Thr Cys Tyr Glu Thr | | |
| 35 40 | | |
| SEQUENCE DESCRIPTION: SEQ ID NO: 87: | | |
| Ser Cys Tyr Phe Ile Pro Asn Glu Gly Val Pro Gly Asp Ser Thr Arg | | |
| 1 5 10 15 | | |
| Lys Cys Met Asp Leu Lys Gly Asn Lys His Pro Ile Asn Ser Glu Trp | | |
| 20 25 30 | | |
| Gln Thr Asp Asn Cys Glu Thr Cys Thr Cys Tyr Glu Thr | | |
| 35 40 45 | | |
| 2) INFORMATION FOR SEQ ID NO: 88: | | |
| | (i) | SEQUENCE CHARACTERISTICS: |

(A) LENGTH: 15
(B) TYPE: AMINO ACID
(C) STRANDEDNESS: SINGLE
(D) TOPOLOGY: LINEAR

| | |
|---|---|
| (ii) | MOLECULE TYPE: |
| (ix) | FEATURE : |

NAME/KEY : Modified site
LOCATION : 1
OTHER INFORMATION : the residue in this position is
either glutamic acid, asparagine, or aspartic acid.

| | |
|---|---|
| (ix) | FEATURE : |

NAME/KEY : Modified site
LOCATION : 4
(D) OTHER INFORMATION : The residue in this position is
either threonine, or serine.

| | |
|---|---|
| (ix) | FEATURE : |

NAME/KEY : Modified site
LOCATION : 6
(D) OTHER INFORMATION : The residue in this position is
either glutamic acid, asparagine, or aspartic acid.

| | |
|---|---|
| (ix) | FEATURE : |

NAME/KEY : Modified site
LOCATION : 8
(D) OTHER INFORMATION : The residue in this position is
either glutamic acid, asparagine, or aspartic acid.

| | |
|---|---|
| (ix) | FEATURE : |

NAME/KEY : Modified site
LOCATION : 9
(D) OTHER INFORMATION : The residue in this position is
either threonine, or serine.

| | |
|---|---|
| (ix) | FEATURE : |

NAME/KEY : Modified site
(B) LOCATION : 11
(D) OTHER INFORMATION : The residue in this position is
either threonine, or serine.

| | |
|---|---|
| (ix) | FEATURE : |

(A) NAME/KEY : Modified site
(B) LOCATION : 13
(D) OTHER INFORMATION : The residue in this position is
either tyrosine, or phenylalanine.

| | |
|---|---|
| (ix) | FEATURE : |

NAME/KEY : Modified site
(B) LOCATION : 14
(D) OTHER INFORMATION : The residue in this position is
either glutamic acid, asparagine, or aspartic acid.

| | |
|---|---|
| (ix) | FEATURE : |

(A) NAME/KEY : Modified site
(B) LOCATION : 15
(D) OTHER INFORMATION : The residue in this position is
either threonine, or serine.

| | |
|---|---|
| (vi) | ORIGINAL SOURCE: |

(A) ORGANISM:

| | |
|---|---|
| (xi) | SEQUENCE DESCRIPTION: SEQ ID NO: 88: |

```
Xaa Trp Gln Xaa Asp Xaa Cys Xaa Xaa Cys Xaa Cys Xaa Xaa Xaa
    1 5 10 15
SEQUENCE DESCRIPTION: SEQ ID NO: 89:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr
                          20 25 30
SEQUENCE DESCRIPTION: SEQ ID NO: 90:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr Glu Trp
                          20 25 30
Gln Thr Asp Asn Cys Glu Thr Cys Thr Cys Tyr Glu Thr
                  35 40 45
SEQUENCE DESCRIPTION: SEQ ID NO: 91:
Glu Trp Gln Thr Asp Asn Cys Glu Thr Cys Thr Cys Tyr Glu Thr Glu
    1 5 10 15
Trp Gln Thr Asp Asn Cys Glu Thr Cys Thr Cys Tyr Glu Thr Glu Trp
                          20 25 30
Gln Thr Asp Asn Cys Glu Thr Cys Thr Cys Tyr Glu Thr Glu Trp Gln
                  35 40 45
Thr Asp Asn Cys Glu Thr Cys Thr Cys Tyr Glu Thr
          50 55 60
SEQUENCE DESCRIPTION: SEQ ID NO: 92:
Glu Tyr Gln Thr Asp Asn Cys Glu Thr Cys Thr Cys Tyr Glu Thr
    1 5 10 15
SEQUENCE DESCRIPTION: SEQ ID NO: 93:
Glu Trp Asn Thr Asp Asn Cys Glu Thr Cys Thr Cys Tyr Glu Thr
1 5 10 15
SEQUENCE DESCRIPTION: SEQ ID NO: 94:
Glu Trp Gln Thr Asp Gln Ser Glu Thr Cys Thr Cys Tyr Asp Thr
    1 5 10 15
SEQUENCE DESCRIPTION: SEQ ID NO: 95:
Trp Gln Thr Asp Asn Cys Glu Thr Cys Thr Cys Tyr Glu Thr
    1 5 10
SEQUENCE DESCRIPTION: SEQ ID NO: 96:
Gln Thr Asp Asn Cys Glu Thr Cys Thr Cys Tyr Glu Thr
    1 5 10
SEQUENCE DESCRIPTION: SEQ ID NO: 97:
Glu Trp Gln Thr Asp Asn Cys Glu Thr Cys Thr Cys
    1 5 10
SEQUENCE DESCRIPTION: SEQ ID NO: 98:
Glu Trp Gln Thr Asp Asn Cys Glu Thr Cys Thr Cys D-Tyr Glu Thr
1 5 10 15
SEQUENCE DESCRIPTION: SEQ ID NO: 99:
Thr-Cys(Acm)-Glu-Asn-Cys(Acm)-Thr-Glu-Thr-Gln-Trp-Cys(Acm)-Glu-Thr-Asp-Tyr
```

1. A method of inhibiting angiogenesis in an individual in need thereof, the method comprising administering to the individual a compound selected from the group consisting of,
a) SEQ ID NO.:5,
b) a SEQ ID NO.:5 derivative able to reduce tube formation in an angiogenesis assay,
c) a SEQ ID NO.:5 fragment able to reduce tube formation in an angiogenesis assay,
d) a SEQ ID NO.:5 analog able to reduce tube formation in an angiogenesis assay, and;
e) combination of any one of a) through d) thereof.

2. The method of claim 1, wherein said compound further comprises a grouping for increasing the stability of said compound.

3. The method of claim 2, wherein said grouping is an acetylaminomethyl moiety attached to a sulfur atom of a cysteine.

4. The method of claim 1, wherein said compound is SEQ ID NO.:7.

5. The method of claim 1, wherein angiogenesis is cancer-associated angiogenesis.

6. The method of claim 1, wherein angiogenesis is metastasis-associated angiogenesis.

7. A method of treating a disease associated with a) phosphorylation of VEGF receptor, b) phosphorylation of PDGF receptor, c) phosphorylation of a substrate mediated by a VEGF receptor, d) phosphorylation of a substrate mediated by a PDGF receptor and combination thereof, the method comprising administering to an individual in need thereof, a compound selected from the group consisting of SEQ ID NO.:5, a SEQ ID NO.:5 derivative able to reduce at least one of substrate phosphorylation by VEGF receptor, substrate phosphorylation by PDGF receptor and substrate phosphorylation by VEGF receptor and PDGF receptor, a SEQ ID NO.:5 fragment able to reduce at least one of substrate phosphorylation by VEGF receptor, substrate phosphorylation by PDGF receptor and substrate phosphorylation by VEGF receptor and PDGF receptor and a SEQ ID NO.:5 analog able to reduce at least one of substrate phosphorylation by VEGF receptor, substrate phosphorylation by PDGF receptor and substrate phosphorylation by VEGF receptor and PDGF receptor.

8. The method of claim 7, wherein said compound further comprises a grouping for increasing the stability of said compound.

9. The method of claim 8, wherein said grouping is an acetylaminomethyl moiety attached to a sulfur atom of a cysteine.

10. The method of claim 7, wherein said compound is SEQ ID NO.:7.

11. The method of claim 10, wherein said disease is an ocular disease.

12. The method of claim 11, wherein said ocular disease is ocular neovascularization.

13. A pharmaceutical composition for treating angiogenesis or an ocular disease, the composition comprising a compound selected from the group consisting of,
a) SEQ ID NO.:5, a SEQ ID NO.:5 derivative able to reduce tube formation in an angiogenesis assay, a SEQ ID NO.:5 fragment able to reduce tube formation in an angiogenesis assay, a SEQ ID NO.:5 analog able to reduce tube formation in an angiogenesis assay and combination thereof, and;
b) a pharmaceutically acceptable carrier.

14. The pharmaceutical composition of claim 13, wherein said compound further comprises a grouping for increasing the stability of said compound.

15. The pharmaceutical composition of claim 14 wherein said grouping is an acetylaminomethyl moiety attached to a sulfur atom of a cysteine.

16. The pharmaceutical composition of claim 15, wherein said compound is SEQ ID NO.:7.

17. A method of regulating, in a cell, an abnormal activation of a molecule selected from the group of molecules consisting of a VEGF receptor, a PDGF receptor, a downstream effector activated by a VEGF receptor and a downstream effector activated by a PDGF receptor, the method comprising contacting the cell with a PSP94 family member.

18. The method of claim 17, wherein the abnormal activation of the VEGF receptor is an increase in the tyrosine kinase signal transduction activity of the VEGF receptor.

19. The method of claim 18, wherein the abnormal activation of the VEGF receptor is promoted by VEGF.

20. The method of claim 17, wherein the abnormal activation of the downstream effector activated by a VEGF receptor is promoted by VEGF.

21. The method of claim 17, wherein the abnormal activation of the PDGF receptor is an increase in the tyrosine kinase signal transduction activity of the PDGF receptor.

22. The method of claim 17, wherein the abnormal activation of the PDGF receptor is promoted by PDGF.

23. The method of claim 17, wherein the abnormal activation of a downstream effector activated by a PDGF receptor is promoted by PDGF.

24. A method for treating a patient having matrix metalloproteinases or pro-matrix metalloproteinases related angiogenesis said method comprising administering to the patient a compound which is a member of the PSP94 family.

25. The method of claim 24, wherein said polypeptide is selected from the group consisting of a MMP-9 and a pro-MMP-9.

26. The method as defined in claim 25, wherein said member of the PSP94 family is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.

27. The method of claim 24, wherein said polypeptide is selected from the group consisting of a MMP-2 and a pro-MMP-2.

28. The method as defined in claim 27, wherein said member of the PSP94 family is selected from the group consisting of;
a) SEQ ID NO.:1,
b) a SEQ ID NO.:1 derivative able to reduce the activity or the level of expression of a polypeptide selected from the group consisting of a pro-matrix metalloproteinase and a matrix metalloproteinase,
c) a SEQ ID NO.:1 fragment able to reduce the activity or the level of expression of a polypeptide selected from the group consisting of a pro-matrix metalloproteinase and a matrix metalloproteinase,
d) SEQ ID NO.:1 analogue able to reduce the activity or the level of expression of a polypeptide selected from the group consisting of a pro-matrix metalloproteinase and a matrix metalloproteinase,
e) SEQ ID NO.:5,
f) a SEQ ID NO.:5 derivative able to reduce the activity or the level of expression of a polypeptide selected from the group consisting of a pro-matrix metalloproteinase and a matrix metalloproteinase,
g) a SEQ ID NO.:5 fragment able to reduce the activity or the level of expression of a polypeptide selected from the group consisting of a pro-matrix metalloproteinase and a matrix metalloproteinase,
h) a SEQ ID NO.:5 analogue able to reduce the activity or the level of expression of a polypeptide selected from the group consisting of a pro-matrix metalloproteinase and a matrix metalloproteinase,
i) SEQ ID NO.:7, and
j) combination of any one of a) through i) thereof.
